SEQUENCE LISTING (99 sequences)

SEQ ID NO: 1 - RNA, 795 nt, Streptomyces hygroscopicus
auggcuaaaaugagaauaucaccggaauugaaaaaacugaucgaaaaauaccgcugcguaaaagauacggaaggaaugucuccugcuaagguauauaagcuggugggagaaaaugaaaaccuauauuuaaaaaugacggacagccgguauaaagggaccaccuaugauguggaacgggaaaaggacaugaugcuauggcuggaaggaaagcugccuguuccaaagguccugcacuuugaacggcaugauggcuggagcaaucugcucaugagugaggccgauggcguccuuugcucggaagaguaugaagaugaacaaagcccugaaaagauuaucgagcuguaugcggagugcaucaggcucuuucacuccaucgacauaucggauugucccuauacgaauagcuuagacagccgcuuagccgaauuggauuacuuacugaauaacgaucuggccgauguggauugcgaaaacugggaagaagacacuccauuuaaagauccgcgcgagcuguaugauuuuuuaaagacggaaaagcccgaagaggaacuugucuuuucccacggcgaccugggagacagcaacaucuuugugaaagauggcaaaguaaguggcuuuauugaucuugggagaagcggcagggcggacaagugguaugacauugccuucugcguccggucgaucagggaggauaucggggaagaacaguaugucgagcuauuuuuugacuuacuggggaucaagccugauugggagaaaauaaaauauuauauuuuacuggaugaauuguuuuag

SEQ ID NO: 2 - RNA, 1026 nt, Streptomyces hygroscopicus
augaaaaagccugaacucaccgcgacgucugucgagaaguuucugaucgaaaaguucgacagcgucuccgaccugaugcagcucucggagggcgaagaaucucgugcuuucagcuucgauguaggagggcguggauauguccugcggguaaauagcugcgccgaugguuucuacaaagaucguuauguuuaucggcacuuugcaucggccgcgcucccgauuccggaagugcuugacauuggggaguuuagcgagagccugaccuauugcaucucccgccgugcacagggugucacguugcaagaccugccugaaaccgaacugcccgcuguucuacaaccggucgcggaggcuauggaugcgaucgcugcggccgaucuuagccagacgagcggguucggcccauucggaccgcaaggaaucggucaauacacuacauggcgugauuucauaugcgcgauugcugauccccauguguaucacuggcaaacugugauggacgacaccgucagugcguccgucgcgcaggcucucgaugagcugaugcuuugggccgaggacugccccgaaguccggcaccucgugcacgcggauuucggcuccaacaauguccugacggacaauggccgcauaacagcggucauugacuggagcgaggcgauguucggggauucccaauacgaggucgccaacaucuucuucuggaggccgugguuggcuuguauggagcagcagacgcgcuacuucgagcggaggcauccggagcuugcaggaucgccacgacuccgggcguauaugcuccgcauuggucuugaccaacucuaucagagcuugguugacggcaauuucgaugaugcagcuugggcgcagggucgaugcgacgcaaucguccgauccggagccgggacugucgggcguacacaaaucgcccgcagaagcgcggccgucuggaccgauggcuguguagaaguacucgccgauaguggaaaccgacgccccagcacucguccgagggcaaagaaauag

SEQ ID NO: 3 - RNA, 552 nt, Streptomyces hygroscopicus
augagcccagaacgacgcccggccgacauccgccgugccaccgaggcggacaugccggcggucugcaccaucgucaaccacuacaucgagacaagcacggucaacuuccguaccgagccgcaggaaccgcaggaguggacggacgaccucguccgucugcgggagcgcuaucccuggcucgucgccgagguggacggcgaggucgccggcaucgccuacgcgggccccuggaaggcacgcaacgccuacgacuggacggccgagucgaccguguacgucuccccccgccaccagcggacgggacugggcuccacgcucuacacccaccugcugaagucccuggaggcacagggcuucaagagcguggucgcugucaucgggcugcccaacgacccgagcgugcgcaugcacgaggcgcucggauaugccccccgcggcaugcugcgggcggccggcuucaagcacgggaacuggcaugacguggguuucuggcagcuggacuucagccugccgguaccgccccguccgguccugcccgucaccgagauuuga

SEQ ID NO: 4 - DNA, 1284 bp, Streptomyces hygroscopicus
atggaatccctgacgttacaacccatcgctcgtgtcgatggcactattaatctgcccggttccaagaccgtttctaaccgcgctttattgctggcggcattagcacacggcaaaacagtattaaccaatctgctggatagcgatgacgtgcgccatatgctgaatgcattaacagcgttaggggtaagctatacgctttcagccgatcgtacgcgttgcgaaattatcggtaacggcggtccattacacgcagaaggtgccctggagttgttcctcggtaacgccggaacggcaatgcgtccgctggcggcagctctttgtctgggtagcaatgatattgtgctgaccggtgagccgcgtatgaaagaacgcccgattggtcatctggtggatgcgctgcgcctgggcggggcgaagatcacttacctggaacaagaaaattatccgccgttgcgtttacagggcggctttactggcggcaacgttgacgttgatggctccgtttccagccaattcctcaccgcactgttaatgactgcgcctcttgcgccggaagatacggtgattcgtattaaaggcgatctggtttctaaaccttatatcgacatcacactcaatctgatgaagacgtttggtgttgaaattgaaaatcagcactatcaacaatttgtcgtaaaaggcgggcagtcttatcagtctccgggtacttatttggtcgaaggcgatgcatcttcggcttcttactttctggcagcagcagcaatcaaaggcggcactgtaaaagtgaccggtattggacgtaacagtatgcagggtgatattcgctttgctgatgtgctggaaaaaatgggcgcgaccatttgctggggcgatgattatatttcctgcacgcgtggtgaactgaacgctattgatatggatatgaaccatattcctgatgcggcgatgaccattgccacggcggcgttatttgcaaaaggcaccaccaggctgcgcaatatctataactggcgtgttaaagagaccgatcgcctgtttgcgatggcaacagaactgcgtaaagtcggcgcggaagtggaagaggggcacgattacattcgtatcactcctccggaaaaactgaactttgccgagatcgcgacatacaatgatcaccggatggcgatgtgtttctcgctggtggcgttgtcagatacaccagtgacgattcttgatcccaaatgcacggccaaaacatttccggattatttcgagcagctggcgcggattagccaggcagcctga

SEQ ID NO: 5 - DNA, 68 bp, Artificial Sequence
  Description of Artificial Sequence Synthetic primer
  modified_base (28)..(47): a, c, t, g, unknown or other
gcggcctctaatacgactcactataggnnnnnnnnnnnnnnnnnnnngttttagagctagaaatagca

SEQ ID NO: 6 - DNA, 82 bp, Artificial Sequence
  Description of Artificial Sequence Synthetic primer
aaaaaagcaccgactcggtgccactttttcaagttgataacggactagccttattttaacttgctatttctagctctaaaac

SEQ ID NO: 7 - DNA, 68 bp, Artificial Sequence; identical to SEQ ID NO: 5
  Description of Artificial Sequence Synthetic primer
  modified_base (28)..(47): a, c, t, g, unknown or other

SEQ ID NO: 8 - DNA, 21 bp, Artificial Sequence
  Description of Artificial Sequence Synthetic primer
aaaaaagcaccgactcggtgc

SEQ ID NO: 9 - DNA, 32 bp, Nicotiana tabacum
ttgctggaggcaagagatgtcctaggtggaaa

SEQ ID NO: 10 - DNA, 32 bp, Nicotiana tabacum
tttccacctaggacatctcttgcctccagcaa

SEQ ID NO: 11 - DNA, 33 bp, Nicotiana tabacum
gctggaggcaagagatgtcctaggtggaaaggt

SEQ ID NO: 12 - DNA, 34 bp, Artificial Sequence
  Description of Artificial Sequence Synthetic oligonucleotide
gctggaggcaagagatgtccttaggtggaaaggt

SEQ ID NO: 13 - DNA, 23 bp, Nicotiana tabacum
gaggcaagagatgtcctaggtgg

SEQ ID NO: 14 - DNA, 24 bp, Artificial Sequence
  Description of Artificial Sequence Synthetic oligonucleotide
gaggcaagagatgtccttaggtgg

SEQ ID NO: 15 - DNA, 22 bp, Artificial Sequence
  Description of Artificial Sequence Synthetic oligonucleotide
gaggcaagagatgtccaggtgg

SEQ ID NO: 16 - DNA, 24 bp, Artificial Sequence
  Description of Artificial Sequence Synthetic oligonucleotide
gaggcaagagatgtcctcaggtgg

SEQ ID NO: 17 - DNA, 24 bp, Artificial Sequence
  Description of Artificial Sequence Synthetic oligonucleotide
gaggcaagagatgtcctaaggtgg

SEQ ID NO: 18 - DNA, 24 bp, Artificial Sequence
  Description of Artificial Sequence Synthetic oligonucleotide
gaggcaagagatgtccttaggtgg

SEQ ID NO: 19 - DNA, 22 bp, Artificial Sequence
  Description of Artificial Sequence Synthetic oligonucleotide
gaggcaagagatgtccaggtgg

SEQ ID NO: 20 - DNA, 24 bp, Artificial Sequence
  Description of Artificial Sequence Synthetic oligonucleotide
gaggcaagagatgtccttaggtgg

SEQ ID NO: 21 - DNA, 24 bp, Artificial Sequence
  Description of Artificial Sequence Synthetic oligonucleotide
gaggcaagagatgtcctcaggtgg

SEQ ID NO: 22 - DNA, 14 bp, Artificial Sequence
  Description of Artificial Sequence Synthetic oligonucleotide
gaggcaataggtgg

SEQ ID NO: 23 - DNA, 24 bp, Artificial Sequence
  Description of Artificial Sequence Synthetic oligonucleotide
gaggcaagagatgtcctaaggtgg

SEQ ID NO: 24 - DNA, 24 bp, Artificial Sequence
  Description of Artificial Sequence Synthetic oligonucleotide
gaggcaagagatgtccttaggtgg

SEQ ID NO: 25 - DNA, 22 bp, Artificial Sequence
  Description of Artificial Sequence Synthetic oligonucleotide
gaggcaagagatgtccaggtgg

SEQ ID NO: 26 - DNA, 23 bp, Nicotiana tabacum
gaggcaagagatgtcctaggtgg

SEQ ID NO: 27 - DNA, 24 bp, Artificial Sequence
  Description of Artificial Sequence Synthetic oligonucleotide
gaggcaagagatgtccttaggtgg

SEQ ID NO: 28 - DNA, 14 bp, Artificial Sequence
  Description of Artificial Sequence Synthetic oligonucleotide
gaggcaataggtgg

SEQ ID NO: 29 - DNA, 22 bp, Artificial Sequence
  Description of Artificial Sequence Synthetic oligonucleotide
gaggcaagagatgtccaggtgg

SEQ ID NO: 30 - DNA, 24 bp, Artificial Sequence
  Description of Artificial Sequence Synthetic oligonucleotide
gaggcaagagatgtccttaggtgg

SEQ ID NO: 31 - DNA, 9 bp, Artificial Sequence
  Description of Artificial Sequence Synthetic oligonucleotide
gaggggtgg

SEQ ID NO: 32 - DNA, 17 bp, Artificial Sequence
  Description of Artificial Sequence Synthetic oligonucleotide
gaggcaagagaaggtgg

SEQ ID NO: 33 - DNA, 24 bp, Artificial Sequence
  Description of Artificial Sequence Synthetic oligonucleotide
gaggcaagagatgtccttaggtgg

SEQ ID NO: 34 - DNA, 24 bp, Artificial Sequence
  Description of Artificial Sequence Synthetic oligonucleotide
gaggcaagagatgtcctaaggtgg

SEQ ID NO: 35 - DNA, 22 bp, Artificial Sequence
  Description of Artificial Sequence Synthetic oligonucleotide
gaggcaagagatgtccaggtgg

SEQ ID NO: 36 - DNA, 22 bp, Artificial Sequence
  Description of Artificial Sequence Synthetic oligonucleotide
gaggcaagagatgtccaggtgg

SEQ ID NO: 37 - DNA, 24 bp, Artificial Sequence
  Description of Artificial Sequence Synthetic oligonucleotide
gaggcaagagatgtcctaaggtgg

SEQ ID NO: 38 - DNA, 82 bp, Nicotiana tabacum
gcaggggcaaataggacaggatttgaaattgaacagaccaagaactggccatccccaacaaactgcagtactcttgcagagg

SEQ ID NO: 39 - DNA, 82 bp, Nicotiana tabacum
cctctgcaagagtactgcagtttgttggggatggccagttcttggtctgttcaatttcaaatcctgtcctatttgcccctgc

SEQ ID NO: 40 - DNA, 82 bp, Nicotiana tabacum; identical to SEQ ID NO: 38

SEQ ID NO: 41 - DNA, 21 bp, Artificial Sequence
  Description of Artificial Sequence Synthetic oligonucleotide
gcaggggcaaatagtcagagg

SEQ ID NO: 42 - DNA, 84 bp, Artificial Sequence
  Description of Artificial Sequence Synthetic oligonucleotide
gcaggggcaaatagtgacaggatttgaaattgaacagaccaagaactggccatccccaacaaactgcagtactcttgtcagagg

SEQ ID NO: 43 - DNA, 84 bp, Artificial Sequence; identical to SEQ ID NO: 42
  Description of Artificial Sequence Synthetic oligonucleotide

SEQ ID NO: 44 - DNA, 71 bp, Artificial Sequence
  Description of Artificial Sequence Synthetic oligonucleotide
gcaggggcaaatagaaattgaacagaccaagaactggccatccccaacaaactgcagtactcttgcagagg

SEQ ID NO: 45 - DNA, 82 bp, Artificial Sequence
  Description of Artificial Sequence Synthetic oligonucleotide
gcaggggcaaatagagacaggatttgaaattgaacagaccaagaactggccatccccaacaaactgcagtactcttcagagg

SEQ ID NO: 46 - DNA, 83 bp, Artificial Sequence
  Description of Artificial Sequence Synthetic oligonucleotide
gcaggggcaaataggacaggatttgaaattgaacagaccaagaactggccatccccaacaaactgcagtactcttgtcagagg

SEQ ID NO: 47 - DNA, 82 bp, Artificial Sequence
  Description of Artificial Sequence Synthetic oligonucleotide
gcaggggcaaatagacaggatttgaaattgaacagaccaagaactggccatccccaacaaactgcagtactcttgacagagg

SEQ ID NO: 48 - DNA, 82 bp, Artificial Sequence
  Description of Artificial Sequence Synthetic oligonucleotide
gcaggggcaaatagtgacaggatttgaaattgaacagaccaagaactggccatccccaacaaactgcagtactcttcagagg

SEQ ID NO: 49 - DNA, 82 bp, Artificial Sequence
  Description of Artificial Sequence Synthetic oligonucleotide
gcaggggcaaatagggacaggatttgaaattgaacagaccaagaactggccatccccaacaaactgcagtactcttcagagg

SEQ ID NO: 50 - DNA, 67 bp, Artificial Sequence
  Description of Artificial Sequence Synthetic oligonucleotide
gcaggggcaaaattgaacagaccaagaactggccatccccaacaaactgcagtactcttgccagagg

SEQ ID NO: 51 - DNA, 80 bp, Artificial Sequence
  Description of Artificial Sequence Synthetic oligonucleotide
gcaggggcaaatagcgacaggatttgaaattgaacagaccaagaactggccatccccaacaaactgcagtactccagagg

SEQ ID NO: 52 - DNA, 82 bp, Artificial Sequence
  Description of Artificial Sequence Synthetic oligonucleotide
gcaggggcaaatagacaggatttgaaattgaacagaccaagaactggccatccccaacaaactgcagtactcttggcagagg

SEQ ID NO: 53 - DNA, 75 bp, Artificial Sequence
  Description of Artificial Sequence Synthetic oligonucleotide
gcaggggcaaatatgacaggatttgaaattgaacagaccaagaactggccatccccaacaaactgcagtcagagg

SEQ ID NO: 54 - DNA, 23 bp, Nicotiana tabacum
gaggcaagagatgtcctaggtgg

SEQ ID NO: 55 - DNA, 22 bp, Artificial Sequence
  Description of Artificial Sequence Synthetic oligonucleotide
gaggcaagagatgtccaggtgg

SEQ ID NO: 56 - DNA, 24 bp, Artificial Sequence
  Description of Artificial Sequence Synthetic oligonucleotide
gaggcaagagatgtccttaggtgg

SEQ ID NO: 57 - DNA, 24 bp, Artificial Sequence
  Description of Artificial Sequence Synthetic oligonucleotide
gaggcaagagatgtcctaaggtgg

SEQ ID NO: 58 - DNA, 24 bp, Artificial Sequence
  Description of Artificial Sequence Synthetic oligonucleotide
gaggcaagagatgtccttaggtgg

SEQ ID NO: 59 - DNA, 24 bp, Artificial Sequence
  Description of Artificial Sequence Synthetic oligonucleotide
gaggcaagagatgtccttaggtgg

SEQ ID NO: 60 - DNA, 24 bp, Artificial Sequence
  Description of Artificial Sequence Synthetic oligonucleotide
gaggcaagagatgtcctcaggtgg

SEQ ID NO: 61 - DNA, 22 bp, Artificial Sequence
  Description of Artificial Sequence Synthetic oligonucleotide
gaggcaagagatgtccaggtgg

SEQ ID NO: 62 - DNA, 24 bp, Artificial Sequence
  Description of Artificial Sequence Synthetic oligonucleotide
gaggcaagagatgtcctaaggtgg

SEQ ID NO: 63 - DNA, 24 bp, Artificial Sequence
  Description of Artificial Sequence Synthetic oligonucleotide
gaggcaagagatgtccttaggtgg

SEQ ID NO: 64 - DNA, 22 bp, Artificial Sequence
  Description of Artificial Sequence Synthetic oligonucleotide
gaggcaagagatgtccaggtgg

SEQ ID NO: 65 - DNA, 24 bp, Artificial Sequence
  Description of Artificial Sequence Synthetic oligonucleotide
gaggcaagagatgtccttaggtgg

SEQ ID NO: 66 - DNA, 24 bp, Artificial Sequence
  Description of Artificial Sequence Synthetic oligonucleotide
gaggcaagagatgtcctaaggtgg

SEQ ID NO: 67 - DNA, 22 bp, Artificial Sequence
  Description of Artificial Sequence Synthetic oligonucleotide
gaggcaagagatgtccaggtgg

SEQ ID NO: 68 - DNA, 24 bp, Artificial Sequence
  Description of Artificial Sequence Synthetic oligonucleotide
gaggcaagagatgtcctaaggtgg

SEQ ID NO: 69 - DNA, 24 bp, Artificial Sequence
  Description of Artificial Sequence Synthetic oligonucleotide
gaggcaagagatgtccttaggtgg

SEQ ID NO: 70 - DNA, 15 bp, Artificial Sequence
  Description of Artificial Sequence Synthetic oligonucleotide
gaggcaagtaggtgg

SEQ ID NO: 71 - DNA, 22 bp, Artificial Sequence
  Description of Artificial Sequence Synthetic oligonucleotide
gaggcaagagatgtccaggtgg

SEQ ID NO: 72 - DNA, 23 bp, Nicotiana tabacum
aaactgcagtactcttgcagagg

SEQ ID NO: 73 - DNA, 22 bp, Artificial Sequence
  Description of Artificial Sequence Synthetic oligonucleotide
aaactgcagtactcttcagagg

SEQ ID NO: 74 - DNA, 24 bp, Artificial Sequence
  Description of Artificial Sequence Synthetic oligonucleotide
aaactgcagtactcttggcagagg

SEQ ID NO: 75 - DNA, 24 bp, Artificial Sequence
  Description of Artificial Sequence Synthetic oligonucleotide
aaactgcagtactcttgtcagagg

SEQ ID NO: 76 - DNA, 24 bp, Artificial Sequence
  Description of Artificial Sequence Synthetic oligonucleotide
aaactgcagtactcttgacagagg

SEQ ID NO: 77 - DNA, 24 bp, Artificial Sequence
  Description of Artificial Sequence Synthetic oligonucleotide
aaactgcagtactcttgtcagagg

SEQ ID NO: 78 - DNA, 22 bp, Artificial Sequence
  Description of Artificial Sequence Synthetic oligonucleotide
aaactgcagtactcttcagagg

SEQ ID NO: 79 - DNA, 22 bp, Artificial Sequence
  Description of Artificial Sequence Synthetic oligonucleotide
aaactgcagtactcttcagagg

SEQ ID NO: 80 - DNA, 24 bp, Artificial Sequence
  Description of Artificial Sequence Synthetic oligonucleotide
aaactgcagtactcttgccagagg

SEQ ID NO: 81 - DNA, 24 bp, Artificial Sequence
  Description of Artificial Sequence Synthetic oligonucleotide
aaactgcagtactcttggcagagg

SEQ ID NO: 82 - DNA, 24 bp, Artificial Sequence
  Description of Artificial Sequence Synthetic oligonucleotide
aaactgcagtactcttgccagagg

SEQ ID NO: 83 - DNA, 20 bp, Artificial Sequence
  Description of Artificial Sequence Synthetic oligonucleotide
aaactgcagtactccagagg

SEQ ID NO: 84 - DNA, 22 bp, Artificial Sequence
  Description of Artificial Sequence Synthetic oligonucleotide
aaactgcagtactcttcagagg

SEQ ID NO: 85 - DNA, 24 bp, Artificial Sequence
  Description of Artificial Sequence Synthetic oligonucleotide
aaactgcagtactcttggcagagg

SEQ ID NO: 86 - DNA, 24 bp, Artificial Sequence
  Description of Artificial Sequence Synthetic oligonucleotide
aaactgcagtactcttgtcagagg

SEQ ID NO: 87 - DNA, 23 bp, Artificial Sequence
  Description of Artificial Sequence Synthetic oligonucleotide
aaactgcagtactcttgcagagg

SEQ ID NO: 88 - DNA, 18 bp, Artificial Sequence
  Description of Artificial Sequence Synthetic oligonucleotide
aaactgcagtaccagagg

SEQ ID NO: 89 - DNA, 20 bp, Artificial Sequence
  Description of Artificial Sequence Synthetic oligonucleotide
aaactgcagtactccagagg

SEQ ID NO: 90 - RNA, 4272 nt, Artificial Sequence
  Description of Artificial Sequence Synthetic polynucleotide
auggacuauaaggaccacgacggagacuacaaggaucaugauauugauuacaaagacgaugacgauaagauggccccaaagaagaagcggaaggucgguauccacggagucccagcagccgacaagaaguacagcaucggccuggacaucggcaccaacucugugggcugggccgugaucaccgacgaguacaaggugcccagcaagaaauucaaggugcugggcaacaccgaccggcacagcaucaagaagaaccugaucggagcccugcuguucgacagcggcgaaacagccgaggccacccggcugaagagaaccgccagaagaagauacaccagacggaagaaccggaucugcuaucugcaagagaucuucagcaacgagauggccaagguggacgacagcuucuuccacagacuggaagaguccuuccugguggaagaggauaagaagcacgagcggcaccccaucuucggcaacaucguggacgagguggccuaccacgagaaguaccccaccaucuaccaccugagaaagaaacugguggacagcaccgacaaggccgaccugcggcugaucuaucuggcccuggcccacaugaucaaguuccggggccacuuccugaucgagggcgaccugaaccccgacaacagcgacguggacaagcuguucauccagcuggugcagaccuacaaccagcuguucgaggaaaaccccaucaacgccagcggcguggacgccaaggccauccugucugccagacugagcaagagcagacggcuggaaaaucugaucgcccagcugcccggcgagaagaagaauggccuguucggaaaccugauugcccugagccugggccugacccccaacuucaagagcaacuucgaccuggccgaggaugccaaacugcagcugagcaaggacaccuacgacgacgaccuggacaaccugcuggcccagaucggcgaccaguacgccgaccuguuucuggccgccaagaaccuguccgacgccauccugcugagcgacauccugagagugaacaccgagaucaccaaggccccccugagcgccucuaugaucaagagauacgacgagcaccaccaggaccugacccugcugaaagcucucgugcggcagcagcugccugagaaguacaaagagauuuucuucgaccagagcaagaacggcuacgccggcuacauugacggcggagccagccaggaagaguucuacaaguucaucaagcccauccuggaaaagauggacggcaccgaggaacugcucgugaagcugaacagagaggaccugcugcggaagcagcggaccuucgacaacggcagcaucccccaccagauccaccugggagagcugcacgccauucugcggcggcaggaagauuuuuacccauuccugaaggacaaccgggaaaagaucgagaagauccugaccuuccgcauccccuacuacgugggcccucuggccaggggaaacagcagauucgccuggaugaccagaaagagcgaggaaaccaucacccccuggaacuucgaggaagugguggacaagggcgcuuccgcccagagcuucaucgagcggaugaccaacuucgauaagaaccugcccaacgagaaggugcugcccaagcacagccugcuguacgaguacuucaccguguauaacgagcugaccaaagugaaauacgugaccgagggaaugagaaagcccgccuuccugagcggcgagcagaaaaaggccaucguggaccugcuguucaagaccaaccggaaagugaccgugaagcagcugaaagaggacuacuucaagaaaaucgagugcuucgacuccguggaaaucuccggcguggaagaucgguucaacgccucccugggcacauaccacgaucugcugaaaauuaucaaggacaaggacuuccuggacaaugaggaaaacgaggacauucuggaagauaucgugcugacccugacacuguuugaggacagagagaugaucgaggaacggcugaaaaccuaugcccaccuguucgacgacaaagugaugaagcagcugaagcggcggagauacaccggcuggggcaggcugagccggaagcugaucaacggcauccgggacaagcaguccggcaagacaauccuggauuuccugaaguccgacggcuucgccaacagaaacuucaugcagcugauccacgacgacagccugaccuuuaaagaggacauccagaaagcccagguguccggccagggcgauagccugcacgagcacauugccaaucuggccggcagccccgccauuaagaagggcauccugcagacagugaaggugguggacgagcucgugaaagugaugggccggcacaagcccgagaacaucgugaucgaaauggccagagagaaccagaccacccagaagggacagaagaacagccgcgagagaaugaagcggaucgaagagggcaucaaagagcugggcagccagauccugaaagaacaccccguggaaaacacccagcugcagaacgagaagcuguaccuguacuaccugcagaaugggcgggauauguacguggaccaggaacuggacaucaaccggcuguccgacuacgauguggaccauaucgugccucagagcuuucugaaggacgacuccaucgacaacaaggugcugaccagaagcgacaagaaccggggcaagagcgacaacgugcccuccgaagaggucgugaagaagaugaagaacuacuggcggcagcugcugaacgccaagcugauuacccagagaaaguucgacaaucugaccaaggccgagagaggcggccugagcgaacuggauaaggccggcuucaucaagagacagcugguggaaacccggcagaucacaaagcacguggcacagauccuggacucccggaugaacacuaaguacgacgagaaugacaagcugauccgggaagugaaagugaucacccugaaguccaagcugguguccgauuuccggaaggauuuccaguuuuacaaagugcgcgagaucaacaacuaccaccacgcccacgacgccuaccugaacgccgucgugggaaccgcccugaucaaaaaguacccuaagcuggaaagcgaguucguguacggcgacuacaagguguacgacgugcggaagaugaucgccaagagcgagcaggaaaucggcaaggcuaccgccaaguacuucuucuacagcaacaucaugaacuuuuucaagaccgagauuacccuggccaacggcgagauccggaagcggccucugaucgagacaaacggcgaaaccggggagaucgugugggauaagggccgggauuuugccaccgugcggaaagugcugagcaugccccaagugaauaucgugaaaaagaccgaggugcagacaggcggcuucagcaaagagucuauccugcccaagaggaacagcgauaagcugaucgccagaaagaaggacugggacccuaagaaguacggcggcuucgacagccccaccguggccuauucugugcuggugguggccaaaguggaaaagggcaaguccaagaaacugaagagugugaaagagcugcuggggaucaccaucauggaaagaagcagcuucgagaagaaucccaucgacuuucuggaagccaagggcuacaaagaagugaaaaaggaccugaucaucaagcugccuaaguacucccuguucgagcuggaaaacggccggaagagaaugcuggccucugccggcgaacugcagaagggaaacgaacuggcccugcccuccaaauaugugaacuuccuguaccuggccagccacuaugagaagcugaagggcucccccgaggauaaugagcagaaacagcuguuuguggaacagcacaagcacuaccuggacgagaucaucgagcagaucagcgaguucuccaagagagugauccuggccgacgcuaaucuggacaaagugcuguccgccuacaacaagcaccgggauaagcccaucagagagcaggccgagaauaucauccaccuguuuacccugaccaaucugggagccccugccgccuucaaguacuuugacaccaccaucgaccggaagagguacaccagcaccaaagaggugcuggacgccacccugauccaccagagcaucaccggccuguacgagacacggaucgaccugucucagcugggaggcgacaaaaggccggcggccacgaaaaaggccggccaggcaaaaaagaaaaaguaa

SEQ ID NO: 91 - RNA, 103 nt, Artificial Sequence
  Description of Artificial Sequence Synthetic polynucleotide
  modified_base (2)..(21): a, c, u, g, unknown or other
gnnnnnnnnnnnnnnnnnnnnguuuuagagcuagaaauagcaaguuaaaauaaggcuaguccguuaucaacuugaaaaaguggcaccgagucggugcuuuuuu

SEQ ID NO: 92 - DNA, 7902 bp, Nicotiana tabacum
  modified_base (6469)..(6469): a, c, t, g, unknown or other
tggctatggtaggaatagccaatcatttttgggagtctagccaaacataaaggccggtccagtgcgagttactgcaaattgagtttggagtgaggattaaaggaagataacatatttccagctaaatagcaaacaaatgacccattaacggaagtggccaaaccaccaaattcaggcatctccaccaaatattagttttttatacacaaaagattcagcaattctttatcaggggtatctttttgtgggtaactgccaaaccaccacaaattttcagttcccactcttaactcttttaacttcaacacaacaacttatttgcttttccttctttgcttatcttgtgcataacgatttcctacaactttagcataatcttggtttgtaatccacaacgtgaaacacaactcctaggcggtttcataccgaggtaagaaatggttttggtttctttggttacatcagctgaatgctttacttgagaaaagctctctctttttcccgtttaggatcttgtttattttctttcgtttttctactcgttaaaattttaacttgattttgtgggtgaaggataactttactcatagtgcgagaacaagttttatatggaccgtaaaagctagaatcttttttacttttgcatataaatttgtgtaataaatgcttaagaaccagaatatttgaaaaagaaaaggaattctacatagtatttaggttcacaagtgggacaatcttcttacactgaaatcaggcttaatttactgctattttgttcagtaaaatgccccaaattggacttgtttctgccgttaatttgagagtccaaggtaattcagcttatctttggagctcgaggtcttctttgggaactgaaagtcaagatggtcacttgcaaaggaatttgttatgttttggtagtagcgactccatggggcataagttaaggattcgtactcccagtgccatgaccagaagattgacaaaggactttaatcctttaaaggtttgttttgaatgcggtgtgattctgaatttatgatcttgggcatatattctctaaaataagagaagtatatcttgccattcaggtagtctgcattgattatccaagaccagagctagacaatacagttaactatttggaggcggcgttattatcatcatcatttcgtacttcctcacgcccaactaaaccattggagattgttattgctggtgcaggtgattttttccagtcatctatatttgtagtcttcatttttcttttttcggagggaagatcattctattagttgtattatcactagaatatttacctgtgcattcttttctgattaactgttttgtaccgcaaaattttaggttcttagttcttcgccattttgcaactaatcagcaagtcccaactgattagattaggagcgatttgaaaattagtttgttttgaactatttttgccgtcactcttatactgttgagttgtcccacatcggtgggatttgaggtccttggtctcacctcataagctagcttttgaggttgagttagacccaatgtccatttatcatggcatccgagccaggcccatcccatttattgttaccgatatggggctcccatttatgttgtccacgctccagtttgcaagcctgggcgtgggggagggggggtggggtgttgagttgtcccatatcggtgggatttgaggtccttggtctccttatatggtcttggacaatcctcgcctcataagctagcttttggggttgagttaggcccaagatccatttatcatatatatgtctattctctcttatcatctgagccatgataagcgggtgaacgtgctgtctattgggtggcatgtccaatggatcattctgaaatattggaggcaaatgaaccaataccttgtgcaagattgatatcacaatacctataatcagagtacttagagttccaaaaatttcaaaacccattgaaaagtcaaacgagttacatataggggttgcactcttctaaggcttgcaatctgtgagaaaaagatgagaaggagatcttcatatttcatctttactaggctggaccattgaccggttagcagttttgaacttgttcttcaacttggcttgcatagtactgtgccgatcatttcttttgtattgtcatcaactggttgattatctgagtacctaaagaaagaatgttatatgcatgatacattctgctgtactataaaagatataataaagaatgctagccgaggtactacatggccttttcagacaaatagaagctgtagcatgattctaattcaatttttttttttttttttttggaatatcaggtttgggtggtttgtctacagcaaaatatctggctgatgctggtcacaaaccgatattgctggaggcaagagatgtcctaggtggaaaggtgaagaatatccatgctttcctttaattttattcctttttcttttgtgtccttccctattgatagtcccttttcaggaaggcttctatttgttttgtttaaaatcatttttcatactctttaaacattcagttgctcaaacaattgcaagggtgttcactattcctatttttgactgtcttcctttctctcagtttagttttattcctctctctctctctctctctatttttggaggaaatagatctgtcctaaaaatttccagctttactactaatagtgttaattgtcgagaaaatagtacagcatattaggtaaaagatatggaaagtatattattattattattattattattattattattattattattattattattattattattattattattattctctattattttaagattgagtcaattttacctgtcctgttggttgcatttctcatataaacattcttttctgtgagatgctatgtgaattagctgatgtttttggtatagagcactatgttagtcagttttatcttactgaagcagtcaccaagaatctagttgtataggctaaaagattgaattagcattaatctttatgtgttctgcacctgaatacttatacctaccttttaggtagctgcatggaaagatgatgatggagattggtatgagactgggttgcacatattctgtaagtttgactcctcaagaatgcatactttaatcttctagtacaacagtttctttcaagatctcttttgtccattaatcagatagctatccctgtttgtcttttgcaaatagccaatatgtcagtcgatctgtattctgccttgcctatctttttttatctgttaatttcgtatggtgactcatacaagttggtgcatctcctttaagttggggcttacccaaatatgcagaacttgtttggagaactagggataaacgatcggttgcagtggaaggaacattcaatgatatttgcgatgcctaacaagccaggggagttcagccgctttgattttcctgaagctcttcctgcgccattaaatggtaagtacttaatcatgagtaaatttctcccttctgcattgattatgtaaacttccccaataaggcatgaaattgattagtccattaatactctggcacattgctaacatcaaaagaacataaaggttcattacgtcttgatcagaatttctgcatgtagctaaagtgattgagtgtctgtataggtttttacacattgcaagcataagccagttatgttatctcttattttcatttctctacctgtatctcttattctcatttctctatctatgcgttattacttctacaggaattttggccatactaaagaacaacgaaatgcttacgtggcccgaaaaagtcaaatttgctattggactcttgccagcaatgcttggagggcaatcttatgttgaagctcaagacggtttaagtgttaaggactggatgagaaagcaagtgcgtgatcattttatcttactctttaaagttcataaccttgaggacatagttgacttgcatgttgttgatttcacatgctagaattgtctacctgcctttctttttctaacaacatacatcttacaaatctcagcagcagctatttgcttaattgcttttcagggtgtgcctgatagggtgacagatgaggtgttcattgccatgtcaaaggcacttaacttcataaaccctgacgagctttcgatgcagtgcattttgattgctttgaacagatttcttcaggttagaatcctgatccaccctcaaaacaaaaagagagaaagggatataatcctaccaaagctgtaaatcatgttagggacctgacatattggtgcaggaaacttatttctgaactttttcactctgtttaacttttctgatatatttgaattattaatctgcaggagaaacatggttcaaaaatggcctttttagatggtaaccctcctgagagactttgcatgccgattgttgaacatattgagtcaaaaggtggccaagtcagactaaactcacgaataaaaaagattgagctgaatgaggatggaagtgtcaaatgttttatactgaataatggcagtacaattaaaggagatgcttttgtgtttgccactccaggtataatatccattatactagcatcgatgcttccagttttcacatttttaatatgaatttataattttttgctgacttttgattatccgattagtggatatcttcaagcttcttttgcctgaagagtggaaagagatcccatatttccaaaagttggagaagctagtgggagttcctgtgataaatgtccatatatggttagtgatgaaaattttacttttcagtgtttgttcttcctctagcatatctatgtatgtgcctgttaatgtctatacgtacatgtttatgtggtcctccggtattctgttaacttcccttgaatgaggaacttatggatgtaagcttttcccaaactttgattggacacattgcaattgtctgttcaactttgatgagcagaactaccattgtttagctattagtggctgagattcctactgaaaagatttgtataaatttaatttgcaggtttgacagaaaactgaagaacacatctgataatctgctcttcagcaggttcatttttgatcaattttattgttccagagcagtttctgcgtgtccatgactacattctcacattagcccccccctccccgcccgccctccttccccggtctcttattaaatacaacatcgtaaattgtggctcttcatcttctctcgtgaacatttgaaagaattttttttgccaattctggtaggtagttttcaaaatcttttatgagcccttacaatttgtttagtactctaccatagtgtttttaatcaataagccaaaggggaaaacctaataaaagtttataaaatttcctcctgtattggtccaattcttttgcaacttatattattaatattatttatcttttggattgaaatggattttgtatatatctaatataaacaaatatgtctcttccgcttatatgatttttcaccatagaaaaatgctcccataaggtcagttattctgtctaaaatatcacacacttcaaccattgagatattttcttctttgcatccagcaatacatttggcatcaatagataggaatccaatgaagatatattatcaatttcctgcaagtttccttggcactagaaacattagatccatatcatgtaaattgcctttgttaaattgaaggtctatgaaatttgggttggttcgaaaaccttttgtttttgcccccccacatccctaatcgtttgttcagtcaaggacagacctgacatgttatgatgaccatttctccaaggcatttataatggactggagtatccatgccacgtttcatcagctacatgttgactatgttcccctacttttttaatggcaccattgttggtggagcaagattatagatgttcctgatacttgtatggttcccttgctcaatctctcttttacttcatgcagaagcccattgctcagtgtgtatgctgacatgtctgttacatgtaaggtattgactcgtctgtaccattcatactggtctaatctgttgggtatgagttgctggtaaattgcataatgcttgttggatatgtgtgtgagttgctgctagatctgtgtcctgctatatttatgtatgagttgctgctattgtaatcttcatttaggatgcataatgatataggttctgtatgtacggaatagtcaggacaatgctcctgtctgtgcacaggggctctacaggaagcaactttcgaaggagaagtaaagaaagagtgatgaaacttaaggcagggaaagtagtttcttttagctaaattttgaaataatttgaaggaggggaaaacgctctctcagtctgtggttgcatttcatactcgcaccaaacggacccttggggggggnaattttgttagaaaatgtgtacaaaatataaagcagtggttactaaaaagttggagaagtagtgggatcttcttgctatttttaacccagaataagacagctatgccatatagctttgattatctgttaacgttctacatataaatagataattaataatgatgtcgtaatactaaagcctggagatcagactgctctaactatcctgagatgattacttttactctcggattagcttaggcgagccgcaagactacattgaatctttagaaatgagaacataaaaaaggtgcagaggtggggaagtggctgaacgatatgcatatgggagtgagtggggagaaaaattatttcctttacttgggtacaatcaagaatggatgacaacttagcccactatatccgttcatgtgttctttagggtcctctgatataactggtctctctgcaggaatattacaaccccaatcagtctatgttggaattggtatttgcacctgcagaagagtggataaatcgtagtgactcagaaattattgatgctacaatgaaggaactagcaaagcttttccctgacgaaatttcggcagatcagagcaaagcaaaaatattgaagtatcacattgtcaaaactccaaggtcagtaatcattttgctttcatagttgtgtaatatgtgagaattacagtccacatggaatatattcctattctgaatcctgattaatctgctttttttctctcaggtctgtttataaaactgtgccaggttgtgaaccctgtcggcccttgcaaagatctcctattgaggggttttatttagctggtgactacacaaaacagaaatacttggcttcaatggaaggtgctgtcttatcaggaaagctttgtgcccaagctattgtacaggtcagttctcacagttgtttttgtccactgatagtatatttgatcaaattttgtcatctttgctgcggtagagaattttagaagcatggacgtcaagcatgcctcttacttataattgctaattctgcgtcagcttatagttctccaaccaatatagtgttaaaccaaaaaaaacaaaattgtgcacacagatcacagagttactcaggatatctgcacttttggagcctcagtagtagcatgataaaatgcagaaggttatgttttttcattccttattaaacttatatctctatattttgcaggattacgagttacttcttggccggagccagaagaagttggcagaagcaagcgtagtttagcatggtgaactaaaatgttgcttctgtacactaaatttaagatgaaggcggccacactgaattagcgttgtacacaacatatacaaggacagtacaacattgaacccaaatacgagaaatgttacacaaatatgaaatatgtgctctgctttccctccga

SEQ ID NO: 93 - DNA, 1749 bp, Nicotiana tabacum
atgccccaaattggacttgtttctgccgttaatttgagagtccaaggtaattcagcttatctttggagctcgaggtcttctttgggaactgaaagtcaagatggtcacttgcaaaggaatttgttatgttttggtagtagcgactccatggggcataagttaaggattcgtactcccagtgccatgaccagaagattgacaaaggactttaatcctttaaaggtagtctgcattgattatccaagaccagagctagacaatacagttaactatttggaggcggcgttattatcatcatcatttcgtacttcctcacgcccaactaaaccattggagattgttattgctggtgcaggtttgggtggtttgtctacagcaaaatatctggctgatgctggtcacaaaccgatattgctggaggcaagagatgtcctaggtggaaaggtagctgcatggaaagatgatgatggagattggtatgagactgggttgcacatattctttggggcttacccaaatatgcagaacttgtttggagaactagggataaacgatcggttgcagtggaaggaacattcaatgatatttgcgatgcctaacaagccaggggagttcagccgctttgattttcctgaagctcttcctgcgccattaaatggaattttggccatactaaagaacaacgaaatgcttacgtggcccgaaaaagtcaaatttgctattggactcttgccagcaatgcttggagggcaatcttatgttgaagctcaagacggtttaagtgttaaggactggatgagaaagcaaggtgtgcctgatagggtgacagatgaggtgttcattgccatgtcaaaggcacttaacttcataaaccctgacgagctttcgatgcagtgcattttgattgctttgaacagatttcttcaggagaaacatggttcaaaaatggcctttttagatggtaaccctcctgagagactttgcatgccgattgttgaacatattgagtcaaaaggtggccaagtcagactaaactcacgaataaaaaagattgagctgaatgaggatggaagtgtcaaatgttttatactgaataatggcagtacaattaaaggagatgcttttgtgtttgccactccagtggatatcttcaagcttcttttgcctgaagagtggaaagagatcccatatttccaaaagttggagaagctagtgggagttcctgtgataaatgtccatatatggtttgacagaaaactgaagaacacatctgataatctgctcttcagcagaagcccattgctcagtgtgtatgctgacatgtctgttacatgtaaggaatattacaaccccaatcagtctatgttggaattggtatttgcacctgcagaagagtggataaatcgtagtgactcagaaattattgatgctacaatgaaggaactagcaaagcttttccctgacgaaatttcggcagatcagagcaaagcaaaaatattgaagtatcacattgtcaaaactccaaggtctgtttataaaactgtgccaggttgtgaaccctgtcggcccttgcaaagatctcctattgaggggttttatttagctggtgactacacaaaacagaaatacttggcttcaatggaaggtgctgtcttatcaggaaagctttgtgcccaagctattgtacaggattacgagttacttcttggccggagccagaagaagttggcagaagcaagcgtagtttag

SEQ ID NO: 94 - DNA, 4610 bp, Nicotiana tabacum
  modified_base (2242)..(2242): a, c, t, g, unknown or other
ggagtaagaattaaaagaagatactactgtagtaacatatttccagctaatagcaaacaaatgacccattaacggaagtggccaaaccaccaaaagcaggcatctccaccaaatattagttttttatatacaaaagattcaacacaaacagttgagtacttctttaatccttcctaattctttgtcaggggtatctttttgtgggtaacggccaaaccaccacaaatttccagttcccacttttaactctttaacttcaattcaactagtttatttccttttgcttcctacaactataacatagtcttggtttcagaatccacaacttgaaatacaactcctaggcggtttcattccgaggtaagaaatgattttgcttttctttggttacatcagctgaatgctttacttgagaaaagctttctctttttttcctttctttgatgtgtaccgcccgtttatgatcttggtatttgctcttttttgtactcgtccggactttaacttgattttgtgggtgaagtattaaaacaagttttatatggactgtaaaagctagaatcttttttacttttgcatataaatttgtataataaatgcttatgaacctgcatataaatttgtataataaatgcatatggccatttgaaaaagaaaaggaattccacatagtatttagattcacaagtgggacaatcttcttacactgaaatatctttatgtcaggcttaatttaccgctattttgctcagtaaaatgccccaaattggacttgtttctgccgttaatttgagagtccaaggtaattcagcttatctttggagctcgaggtcttcgttgggaactgaaagtcaagatggtcgcttgcaaaggaatttgttatgttttggtagtagcgactccatggggcataagttaaggattcgtactcccagtgccacgaccagaagattgacaaaggactttaatcctttaaaggtttgttttgaatgcgaaagtgtgatgatggatttatggtcatgggcatatattctctaaaataagagaagtatatcttgccattcaggtagtctgcattgattatccaagaccagagctagacaatacagttaactatttggaggcggcgttattatcatcatcatttcgtacttcctcacgcccaactaaaccattggagattgttattgctggtgcaggtgattttttccggtcatctatatttgtagtcttcatttctctttcttcggaaggaagatcattctattagttgtattatcactagaatatttacctgggcattcttttctgattaactgttttggaccgcaaaattttaggttcttacttcttcgccattttgcaactaatcaacaagtcccagctgattagattaggagcggtttgaaaattcgtttgttttgatctatttttgccgtcactccatttatatacatatctattctctcttatcatctgagccatgataagcaggtgaacgtgctgtctattgggtggcatgtccaaaggatcattctgaaatattggaggctaatgaatcaatatcttgtgcaagattgatctcactatacctataatcagagtacttagagttccaaaaatttcaaaaccaattgaaaagtcaaacgagttacatataggggttgcactcttctaaggcttgcaatctgtgagaaaaagatgagaaggagatcttcatatttcatctttactagtctggaccactgaccggttagcagttttcaacgtgttcttcaacttggcttgcaaaatactgtgccggtcatttctcttgtattgtcatcaactggttgattatctgagtacctaaagaaagaatgttatatgcatgattcattctgctgtactataaaagatataataaagaatgctagccgaggtactacatagccttttccaataaatagaagctgtaacatgattctaattcattttttttgcaatatcaggtttgggtggtttgtctacagcaaaatatctggcagatgctggtcacaaaccgatattgctggaggcaagagatgtcctaggtggaaaggtgaagaagatccagtctttcctttaattttattcctttttcttttgtgtccttccctattgatagtcccttttcaggaaggcttctgtttgttttatttaaaatcatttttcatactctttaaacattcagttgctcaaacaattgcaagggtgttcactnttactactaataaagttggttgtcgaaaaattatacagcatattaggtaaaagatatggaaagtatattattatcattctctattattttatgattcagtcaattttacccgtcctgttgggtgtatttctcgtataaatacagtcttatctgtgagatgctatgtgaattagctgttgttttcagtatagaacactatcttagtctgttttatcttactgaagcagtcaccaagaatctagctgtataggctaaaatattgaattagcattaatctttatctgttctgcacctgaatacttatacctcccttttaggtagctgcatggaaagatgatgatggagattggtatgagactgggttgcatatattctgtaagtttgactcctcaagaatgcgtactttaatcttctaatacagtcatagcaatttctttcaagatcttttttgtccattaatcagatagcaatccctgtttgtcttttgttttttgcaaataaccaatttttgtcagtcgatctgtattctgccttgcctctcttttttcatctgttaatttcgtatggtgactcatacaagttggtgcatctcctttaagttggggcttacccaaatatgcagaacctgtttggagaactagggattaacgatcgattgcagtggaaggaacattcaatgatatttgcgatgcctaacaagccaggggaattcagccgctttgattttcctgaagctcttcctgcgccattaaatggtaagtacttaatcatgagtaaattttttcccttctgtattgaatattgattatgcaaacttccccagtaaggtatgaaattgattagtccattaatacctctggcgcattgctaacattaaaagaacataaaggttcattacgtcttgatcagaatttctgcatgtagctaaagtgattgagtgtatgtacaaatttttacacattgcaagcataagccagttattgttatctcttattttcatttctctagctgtatctcttattctcatttctctatctatgtgttattacttctacaggaattttggccatactaaagaacaatgaaatgcttacatggcccgaaaaagtcaaatttgctattggactcttgccagcaatgcttggagggcaatcttatgttgaagctcaagacggtttaagtgttaaggactggatgagaaagcaagtgcgtgatcattttatcttactcttttagctcataaccttgaagacatagttgacttgcaagttgttgatttaacatgttagaaatgtctacctgcctttccttttctaacaacatacatcttgcaaaactcatcagcagctatttgcttaattgcttttcagggtgtgcctgatagggtgacagatgaggtgttcattgccatgtcaaaggcacttaacttcataaaccctgacgagctttcgatgcagtgcattttgattgctttgaacagatttcttcaggttagaatcctgatccaccctcaaaacaaaaagagagaaagggatatagtcctaccaaagctgtaaatcatgtttgggacctgacatattggtgcaggaaacttatgagtgaacttgtccactcagtttaacttttctgatatatttgaattatcaatttgcaggagaaacatggttcaaaaatggcctttttagatggtaaccctcctgagagactttgcatgccgattgttgaacatattgagtcaaaaggtggccaagtcagactaaactcacgaataaaaaagattgagctcaatgaggatggaagtgtcaaatgttttatactgaataatggcagtacaattaaaggagatgcttttgtgtttgccactccaggtataatatccattatactagtatcgatgcttccagttttcacatttttaatatgaatttataaatttttgctggcttttgattatccgattagtggatatcttcaagcttcttttgcctgaagactggaaagagatcccatatttccaaaagttggagaagctagtgggagttcctgtgataaatgtccatatatggttagtgatgaaaattttacttgtcagtgtttgttcttcctctagcatatctatgtatgtgcgtgtaaatgtctatacgtacataaacatgtagtcctctgtttttgtgttaacttccattgaatgaggaacttatggatgtacgcttttccaaaactttgattggacacattgctttgtgttttcaactttgatgagcagaactaccattgtttagctattagtggttgagattcctattgaaaagatttgtataaatttaatttgcaggtttgacagaaaactgaagaacacatctgataa

SEQ ID NO: 95 - DNA, 1344 bp, Nicotiana tabacum
atgccccaaattggacttgtttctgccgttaatttgagagtccaaggtaattcagcttatctttggagctcgaggtcttcgttgggaactgaaagtcaagatggtcgcttgcaaaggaatttgttatgttttggtagtagcgactccatggggcataagttaaggattcgtactcccagtgccacgaccagaagattgacaaaggactttaatcctttaaaggtagtctgcattgattatccaagaccagagctagacaatacagttaactatttggaggcggcgttattatcatcatcatttcgtacttcctcacgcccaactaaaccattggagattgttattgctggtgcaggtttgggtggtttgtctacagcaaaatatctggcagatgctggtcacaaaccgatattgctggaggcaagagatgtcctaggtggaaaggtagctgcatggaaagatgatgatggagattggtatgagactgggttgcatatattctttggggcttacccaaatatgcagaacctgtttggagaactagggattaacgatcgattgcagtggaaggaacattcaatgatatttgcgatgcctaacaagccaggggaattcagccgctttgattttcctgaagctcttcctgcgccattaaatggaattttggccatactaaagaacaatgaaatgcttacatggcccgaaaaagtcaaatttgctattggactcttgccagcaatgcttggagggcaatcttatgttgaagctcaagacggtttaagtgttaaggactggatgagaaagcaaggtgtgcctgatagggtgacagatgaggtgttcattgccatgtcaaaggcacttaacttcataaaccctgacgagctttcgatgcagtgcattttgattgctttgaacagatttcttcaggagaaacatggttcaaaaatggcctttttagatggtaaccctcctgagagactttgcatgccgattgttgaacatattgagtcaaaaggtggccaagtcagactaaactcacgaataaaaaagattgagctcaatgaggatggaagtgtcaaatgttttatactgaataatggcagtacaattaaaggagatgcttttgtgtttgccactccagtggatatcttcaagcttcttttgcctgaagactggaaagagatcccatatttccaaaagttggagaagctagtgggagttcctgtgataaatgtccatatatggtttgacagaaaactgaagaacacatctgataatctgctcttcagcaggttcatttttgatcaattttattgttccagagcagtttctgcgtgtccatga

SEQ ID NO: 96 - DNA, 3047 bp, Nicotiana tabacum
tctctctttctgtctctattgttccgtctctttctcctgaatcttgccaaattccttgtcttcagctcccccccttaaaagtcacccattttagttattctccctattcctctctcttttacttttaccattacaatataagccttttcttactttgaaacccgattgaccactaatagttaattcttgtaacttaaaaagatcacctccattctctttctctaagttcccttcttttccttttcagtaactccaaaacatccaccacttgaactaattatataagccccatctcttaataattaagaagcccttgttttgcaaccttttatttaattagagataaaaaaaaatgtggatgatgggttacaacgacgggggagatttcaacatgcaggattccttcaatggaagaaagcttcgtccactaatgccaagagtccctcatcttcccactgctaatatttctactaatccaacttgtttaagaagtattcatggcgaaaattttattgcacttaatcaccatcagcttggtaagtaattaaatgctagagattatatatatactatatagagttgtctttctacttttctcttttttccctttccgttcctttttttgcatgcgtgacattgatactttgtcttacttcgtatatcaaaaaagtcatatatcaacagatctaacgactagccatatttataatttaatggttacaatttttcttttcacctcagctatgagtgagcaaaataagagagattttaatacacaacaattagttgtgagctcacgttggaatccaactccagaacaacttcaaaccctcgaggagttgtatcgacgtggcacgagaactccttctgctgaacagattcagcatattactgctcaacttcgacgttatgggaaaattgaaggcaagaatgttttttactggtttcaaaatcacaaggctagggaacgccaaaaacgacgccgtcaacttgaatctgctgctgctgctgctggtggcgatgatcagtctcgtagtatttgtaacgcagaaaacaccggaaggaaagaatcaggtatacgttaatccatttgcttgtgtatagtttaagccaggtcaaaatcagaaatttatcaaaggatttataatgatatatatatatatatatatataaaaggattttcagtacaattttctagcgaaggagatttagttgaaccccttcaatgaacatggctccgccgcatgatgattttattcaactcacaaatatttatttaaactccaaatatttatacatgtgaaaatcttgaaaattagaacaagtattatgttataaatcttgaaggggtggtgcacaacatgcaacttaacaaaaagacatgcaatgagaatgatacatgaaagagaaatttgtttgatagcgtgtgatgatacattaaatattcttttattcttttccaatccatccccactcgcattttgttgttatttaagatgtaaagttgcattcgtgtttggattttgagagatatgattcttcttccattcggattgttgcttgagatataaaaagtttctttactcttttgtttttgtgtttgtcgtcgatcttgcaggggcaaataggacaggatttgaaattgaacagaccaagaactggccatccccaacaaactgcagtactcttgcagaggtccttaattccctatatatttttcactttgcctttttttttctctctccttagactctccattctcattttcataaccaaaaaagcaattagaattccttaaacaggccattatgctgttatgattacaagatcatagaaaaaatatagatattgttttgttttttcaaaaaaaaaaaaaaaaaagtctaagatgtaaaaggaagatctatttattgatcacgccgtagctaggcagtaaattaacaaaagcaatatttcttgggaaatataaaagggtccaaaagctgtgattcttcctttctgttggggctgccaattctgtacgtacgtagctagttgaatgaatagctaaatagctagggttttgggattttgtgcgtttacattttattagacaagtgatatgggcacccatgatccatcctatcacgaaaagacatttccaaaccctcttcagtgatatttgactttccatatagcgtaggtaaaatgctctatcaaatcaggaagtaatgatattcatatatagagaaactaaacattttgctgtgcctatgaacaatggtcaaactagggttgttacttttcactcgagaaagataataaacgttgcatgaaatagtggtaacaaggaaattgatgatttcttttttgcattgcaataaatttacagaaaactgtggcaacaacaaaagcagcaggagtggcagaatgtagaatagcagcagaaagatggataccactcgatgaaggagaacaaagaaggagcctattactagctgatcaaaggaatgccacgtggcagatgatgcatttgtcttgttcacctcccacctccaccaccccccatcatcatctcatgaacatcaactcatctactgcatcaatcagtaatactattactactccaataatttgcagtagtagtccatcaacaccaagaacgacaatggagccaaagcaactcttcaaaaccaaagatcatctcaacatttttatagcacccttcagaacagaaaataacaaacacgaaaatatcgaaaacaatcttggagatgttggccaggaagaatctcagacccttgaattgtttcctctccgcagcagcaacgacgataataatctttcagagaaggatgaagtagagatatcaggggccgatgctaactcgaatagtaactttagtggttctcattaccagttttttgagtttcttccacttaaaaactaagggatctagagaccgggtgctttaatttcttagtctcttcctatttatgtaacttaaaattaatgatgggatttcccccctattattagtaatgttgtggttcaatgatatatgtatc

SEQ ID NO: 97 - DNA, 1152 bp, Nicotiana tabacum
atgtggatgatgggttacaacgacgggggagatttcaacatgcaggattccttcaatggaagaaagcttcgtccactaatgccaagagtccctcatcttcccactgctaatatttctactaatccaacttgtttaagaagtattcatggcgaaaattttattgcacttaatcaccatcagcttgctatgagtgagcaaaataagagagattttaatacacaacaattagttgtgagctcacgttggaatccaactccagaacaacttcaaaccctcgaggagttgtatcgacgtggcacgagaactccttctgctgaacagattcagcatattactgctcaacttcgacgttatgggaaaattgaaggcaagaatgttttttactggtttcaaaatcacaaggctagggaacgccaaaaacgacgccgtcaacttgaatctgctgctgctgctgctggtggcgatgatcagtctcgtagtatttgtaacgcagaaaacaccggaaggaaagaatcaggggcaaataggacaggatttgaaattgaacagaccaagaactggccatccccaacaaactgcagtactcttgcagagaaaactgtggcaacaacaaaagcagcaggagtggcagaatgtagaatagcagcagaaagatggataccactcgatgaaggagaacaaagaaggagcctattactagctgatcaaaggaatgccacgtggcagatgatgcatttgtcttgttcacctcccacctccaccaccccccatcatcatctcatgaacatcaactcatctactgcatcaatcagtaatactattactactccaataatttgcagtagtagtccatcaacaccaagaacgacaatggagccaaagcaactcttcaaaaccaaagatcatctcaacatttttatagcacccttcagaacagaaaataacaaacacgaaaatatcgaaaacaatcttggagatgttggccaggaagaatctcagacccttgaattgtttcctctccgcagcagcaacgacgataataatctttcagagaaggatgaagtagagatatcaggggccgatgctaactcgaatagtaactttagtggttctcattaccagttttttgagtttcttccacttaaaaactaa

SEQ ID NO: 98 - DNA, 65 bp, Artificial Sequence
  Description of Artificial Sequence Synthetic oligonucleotide
  modified_base (28)..(44): a, c, t, g, unknown or other
gcggcctctaatacgactcactataggnnnnnnnnnnnnnnnnngttttagagctagaaatagca

SEQ ID NO: 99 - DNA, 65 bp, Artificial Sequence; identical to SEQ ID NO: 98
  Description of Artificial Sequence Synthetic oligonucleotide
  modified_base (28)..(44): a, c, t, g, unknown or other

(19) United States
(12) Patent Application Publication
(10) Pub. No.: US 2018/0201944 A1
(43) Pub. Date: Jul. 19, 2018

(54) DNA-FREE GENOME EDITING AND SELECTION METHODS IN PLANTS

(71) Applicant: Noble Research Institute, LLC, Ardmore, OK (US)

(72) Inventors: Zengyu Wang, Ardmore, OK (US); Miao Chen, Ardmore, OK (US)

(21) Appl. No.: 15/873,699

(22) Filed: Jan. 17, 2018

Related U.S. Application Data
(60) Provisional application No. 62/447,115, filed on Jan. 17, 2017.

(51) Int. Cl. C12N 15/82; C12N 9/22; C12N 15/11

(52) U.S. Cl. CPC C12N 15/8209; C12N 9/22; C12N 2800/80; C12N 2310/20; C12N 15/11

(57) ABSTRACT

Clustered regularly interspaced short palindromic repeats (CRISPR) and the CRISPR associated protein 9 (Cas9) offer an effective way of creating targeted mutagenesis in plants. To alleviate concerns related to genetically modified plants, the present disclosure provides a novel and efficient genome editing system that allows the regeneration of mutant plants without DNA stable integration. This DNA free system utilizes Cas9 mRNA, guide RNA and selectable marker RNA to infect plant protoplasts. After a short period of selection of the transfected cells, non-transgenic plants carrying expected mutations were regenerated. The system offers a way of creating desired mutants without transgenic elements.

### CROSS-REFERENCE TO RELATED APPLICATIONS

The present application claims priority to U.S. Provisional Application No. 62/447,115, filed on Jan. 17, 2017 which is herein incorporated by reference in its entirety.

### COLOR DRAWINGS

The patent or application file contains at least one drawing executed in color. Copies of this patent or patent application publication with color drawing(s) will be provided by the Office upon request and payment of the necessary fee.

### SEQUENCE LISTING

The instant application contains a Sequence Listing which has been filed electronically in ASCII format and is hereby incorporated by reference in its entirety. Said ASCII copy, created on Jan. 17, 2018, is named 16-21023-US SL.txt and is 59,773 bytes in size.

### BACKGROUND

In recent years, clustered regularly interspaced short palindromic repeats (CRISPR) and CRISPR associated protein 9 (Cas9) have been developed into an effective genome editing system. The introduction of CRISPR/Cas9 into cells generates DNA double-strand breaks (DSBs), which are typically repaired by error-prone non-homologous end-joining (NHEJ) resulting in nonspecific insertions or deletions (indels). The CRISPR/Cas9 system has been successfully used to create targeted mutations in many organisms, including model and crop plants such as Arabidopsis, tobacco, tomato, rice and maize.

In general, the CRISPR/Cas9 system employs a plasmid DNA to transfect the target plant cells. The plasmid DNA can be introduced into plant cells by Agrobacterium-mediated transformation, polyethylene glycol (PEG) or electroporation treatment of protoplasts, particle bombardment or other methods. To create mutant plants, a selectable marker gene is also needed to select for transformed cells. The CRISPR/Cas9 plasmid DNA and the selectable marker gene will be stably integrated in the plant genome. Thus the regenerated mutant plants contain transgenic elements.

The cost of meeting regulatory requirements is a substantial impediment to the commercialization of transgenic crops. Thus, an alternative approach is needed to efficiently produce mutant plants without the introduction and integration of foreign DNA. The present disclosure provides RNA-based approaches to genome editing and selection thereby alleviating many of the concerns associated with traditional transgenic plants.

### SUMMARY

In some embodiments, the present disclosure relates to a method for selecting cells carrying a transfected nucleic acid. The method comprises a first step of exposing a plurality of cells to a first RNA molecule under conditions sufficient to promote transfection of the first RNA molecule into one or more of the plurality of cells. The first RNA molecule or a peptide or protein encoded by the first RNA molecule permits selection of the one or more of the plurality of cells transfected with the first RNA molecule based on a first selectable property. In one embodiment, the present disclosure relates to a method for selecting cells carrying a transfected nucleic acid. The method comprises a first step of exposing a plurality of cells to a first RNA molecule under conditions sufficient to promote transfection of the first RNA molecule into one or more of the plurality of cells. The first RNA molecule encodes a peptide or protein that acts to reduce the activity of a selection agent. In this instance, the selection agent is sufficient to kill, inhibit or reduce proliferation of cells that have not been transfected with the first RNA molecule. Following transfection of the first RNA molecule, a second step is performed where the plurality of cells are exposed to the selection agent. Following a suitable exposure to the selection agent, colonies or groups of cells that survive and proliferate in the presence of the selection agent are selected as indicative of a successful transfection.

The method may further comprise exposing the plurality of cells to one or more additional nucleic acids under conditions sufficient to transfect the plurality of cells with the one or more additional nucleic acids. In this instance, the additional nucleic acids are either sufficient to edit a genomic region of the transfected cells or stably integrate into the genome of the transfected cells. Thus, the additional nucleic acids can be either RNA or DNA. In certain aspects, the additional nucleic acids comprise a second RNA molecule and a third RNA molecule. For example, the second RNA molecule encodes for Cas9 and the third RNA molecule is a guide RNA molecule comprising a target sequence and a scaffold sequence for Cas9 binding, wherein the target sequence defines the genomic region to be edited.

It should be understood that the above methods may comprise one or more additional first RNA molecules for providing resistance to one or more additional selection agents. Similarly, multiple species of additional nucleic acids may be used for editing multiple genomic regions.

Following the selection step, the methods may further comprise subjecting the selected resistant cells to conditions sufficient to promote growth of the selected cells into a plant.

The present disclosure is also directed to kits for transfecting plants cells with selection nucleic acids. In one embodiment, the kit comprises a first RNA molecule and a selection agent as described above. Additionally, the kit may comprise one or more reagents to promote transfection of the first RNA molecule. The kit may further comprise additional nucleic acids for genome editing such as the second and third RNA molecules described above or other nucleic acids such as a plasmid DNA for stable integration into the genome of the cells. In some embodiments, the kit can include a first RNA molecule, the first RNA molecule or a peptide or protein encoded by the first RNA molecule being sufficient to produce a first selectable property in one or more of a plurality of cells, and a tranfection reagent. The kit may further comprise additional nucleic acids for genome editing such as the second and third RNA molecules described above or other nucleic acids such as a plasmid DNA for stable integration into the genome of the cells.

The present disclosure further provides for genetically-modified plants produced without integration of exogenous DNA into their genomes. For example, genetically-modified plants generated using the methods described herein. Seeds produced by these genetically-modified plants are also provided.

Other objects, features and advantages of the present disclosure will become apparent from the following detailed description. It should be understood, however, that the detailed description and specific examples, while indicating specific embodiments of the invention, are given by way of illustration only, since various changes and modifications within the scope of the present disclosure will become apparent to those skilled in the art from this detailed description.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the disclosure is not limited to specific methods and instrumentalities disclosed herein.

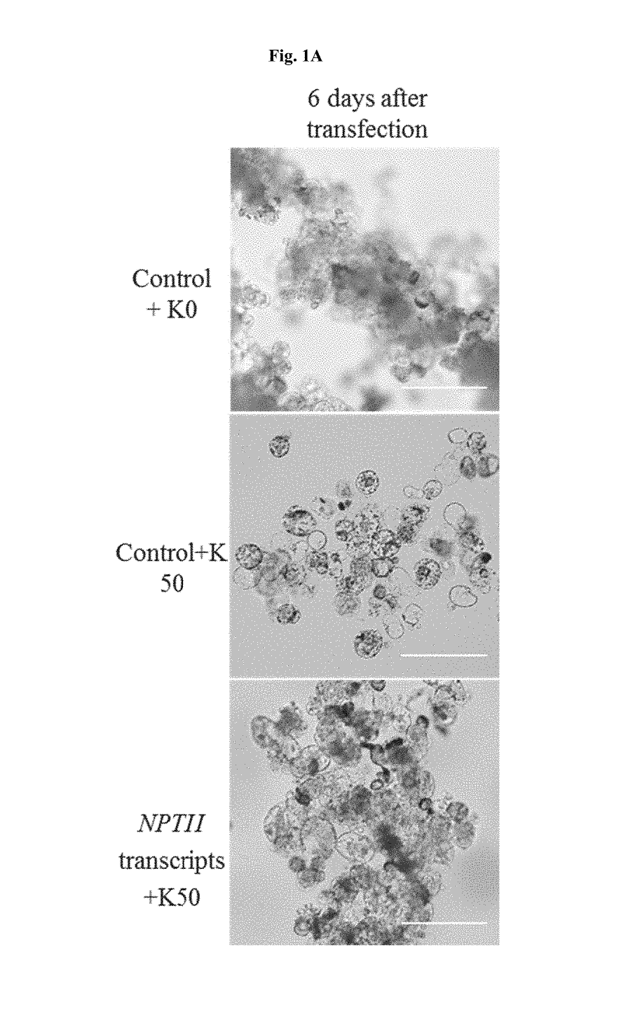

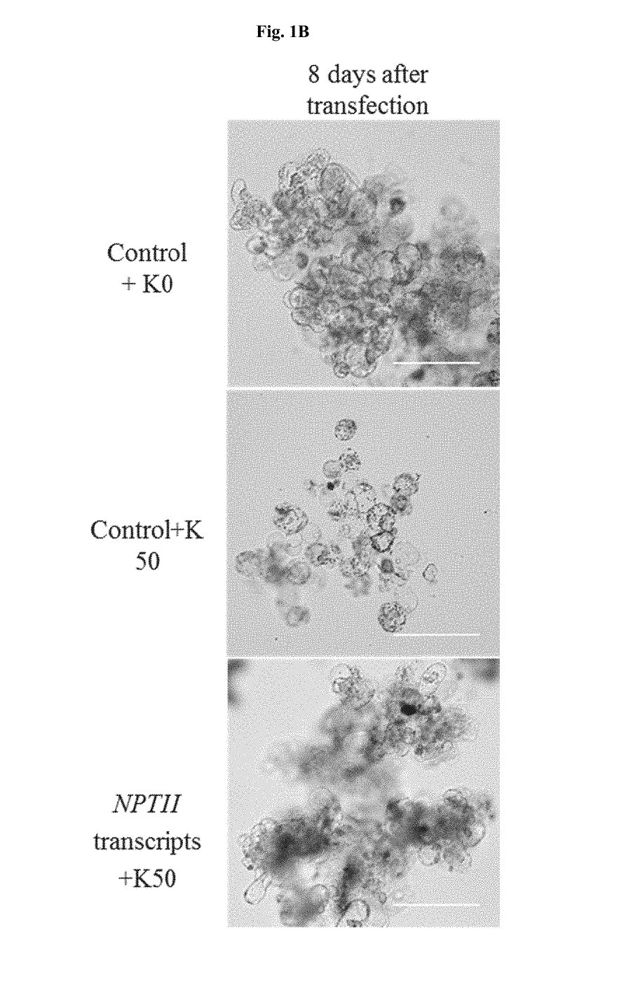

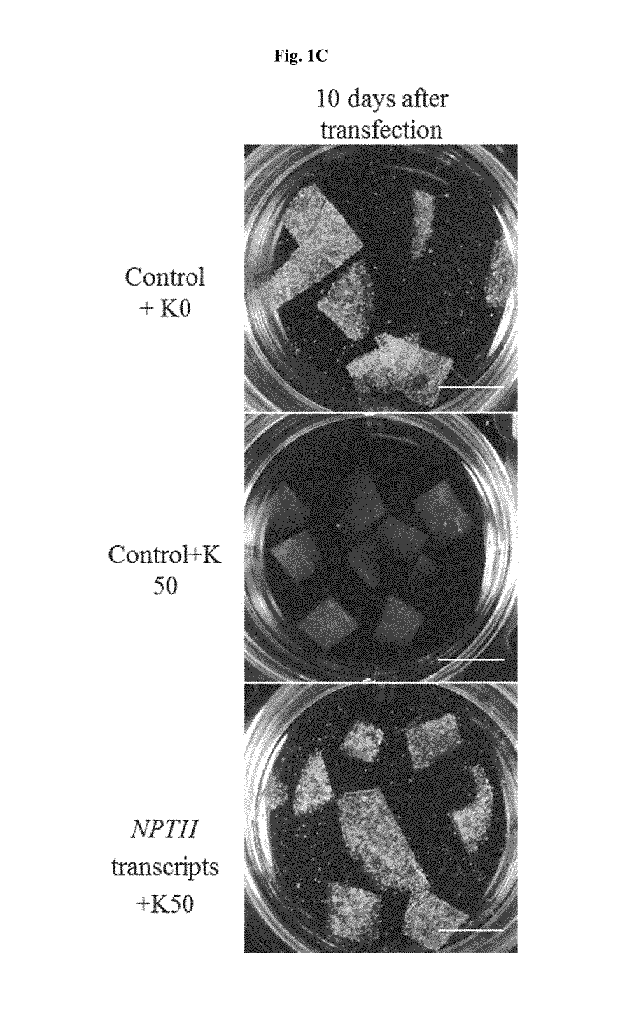

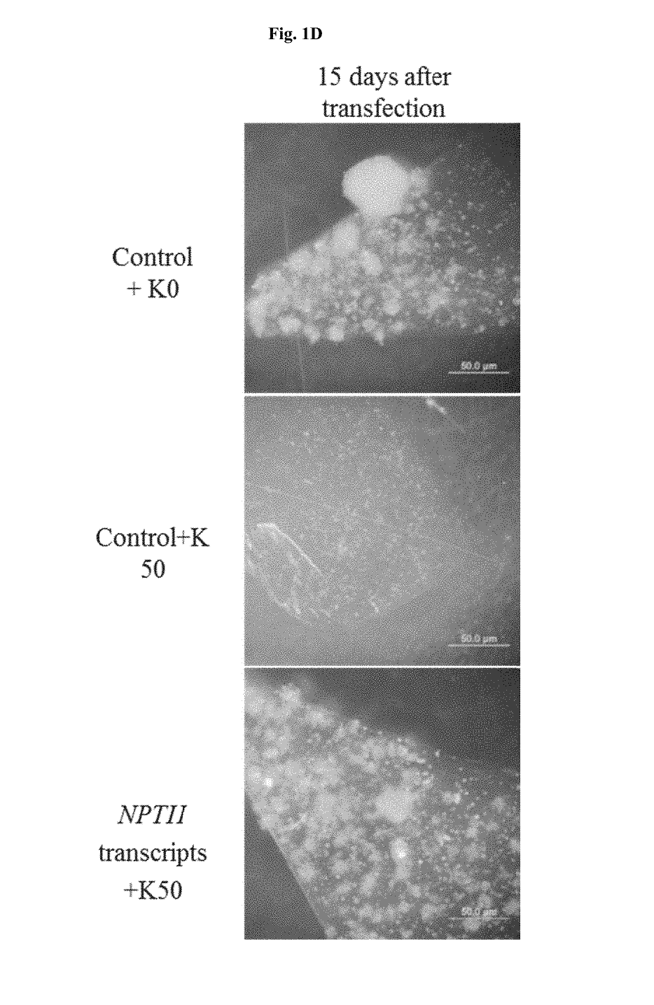

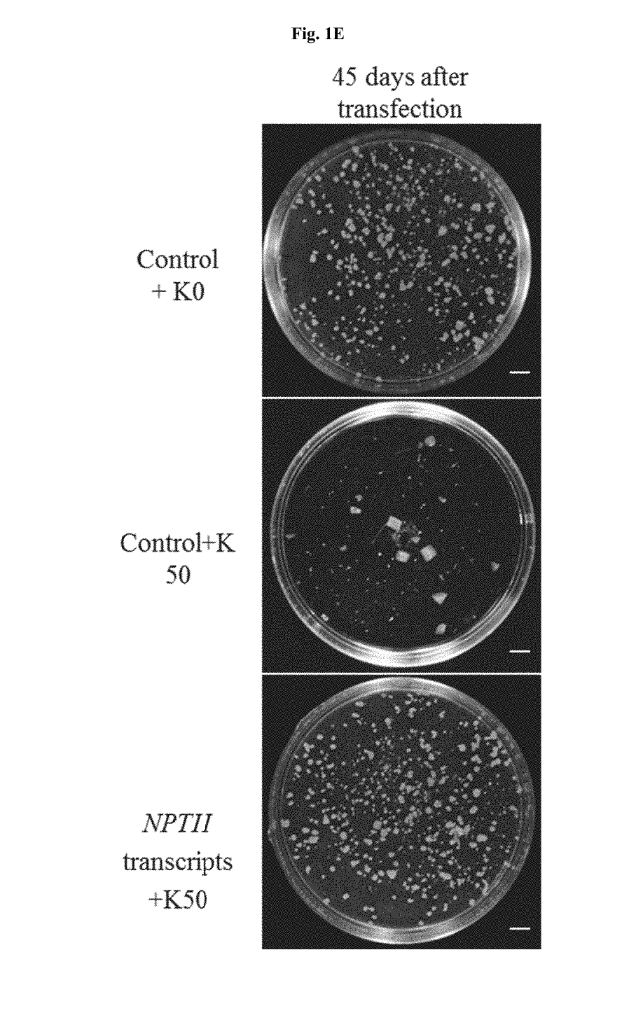

FIG. 1A depicts cell growth for the short-term non-transgenic selection system of the present disclosure in tobacco protoplasts 6 days post-transfection. Growth of tobacco protoplasts was inhibited by 6-day 50 mg/l kanamycin selection. Control: tobacco protoplasts were transfected with sterile water instead of NPTII transcripts. K0: no kanamycin present. +K50: 50 mg/l kanamycin present. Colonies were formed without NPTII transcripts and kanamycin (Control+K0). Without NPTII transcripts, colony formation was blocked by the presence of kanamycin (Control+K50). Kanamycin resistant colonies were obtained when protoplasts were transfected with NPTII transcripts (NPTII transcripts+K50). Scale bar=25 μm.

FIG. 1B depicts cell growth for the short-term non-transgenic selection system of the present disclosure in tobacco protoplasts 8 days post-transfection. Control: tobacco protoplasts were transfected with sterile water instead of NPTII transcripts. K0: no kanamycin present. +K50: 50 mg/l kanamycin present. Colonies were formed without NPTII transcripts and kanamycin (Control+K0). Without NPTII transcripts, colony formation was blocked by the presence of kanamycin (Control+K50). Kanamycin resistant colonies were obtained when protoplasts were transfected with NPTII transcripts (NPTII transcripts+K50). Scale bar=25 μm.

FIG. 1C depicts cell growth for the short-term non-transgenic selection system of the present disclosure in tobacco protoplasts 10 days post-transfection. Control: tobacco protoplasts were transfected with sterile water instead of NPTII transcripts. K0: no kanamycin present. +K50: 50 mg/l kanamycin present. Colonies were formed without NPTII transcripts and kanamycin (Control+K0). Without NPTII transcripts, colony formation was blocked by the presence of kanamycin (Control+K50). Kanamycin resistant colonies were obtained when protoplasts were transfected with NPTII transcripts (NPTII transcripts+K50). Scale bar=1 cm.

FIG. 1D depicts cell growth for the short-term non-transgenic selection system of the present disclosure in tobacco protoplasts 15 days post-transfection. Control: tobacco protoplasts were transfected with sterile water instead of NPTII transcripts. K0: no kanamycin present. +K50: 50 mg/l kanamycin present. Colonies were formed without NPTII transcripts and kanamycin (Control+K0). Without NPTII transcripts, colony formation was blocked by the presence of kanamycin (Control+K50). Kanamycin resistant colonies were obtained when protoplasts were transfected with NPTII transcripts (NPTII transcripts+K50). Scale bar=50 μm.

FIG. 1E depicts cell growth for the short-term non-transgenic selection system of the present disclosure in tobacco protoplasts 45 days post-transfection. Control: tobacco protoplasts were transfected with sterile water instead of NPTII transcripts. K0: no kanamycin present. +K50: 50 mg/l kanamycin present. Colonies were formed without NPTII transcripts and kanamycin (Control+K0). Without NPTII transcripts, colony formation was blocked by the presence of kanamycin (Control+K50). Kanamycin resistant colonies were obtained when protoplasts were transfected with NPTII transcripts (NPTII transcripts+K50). Scale bar=1 cm.

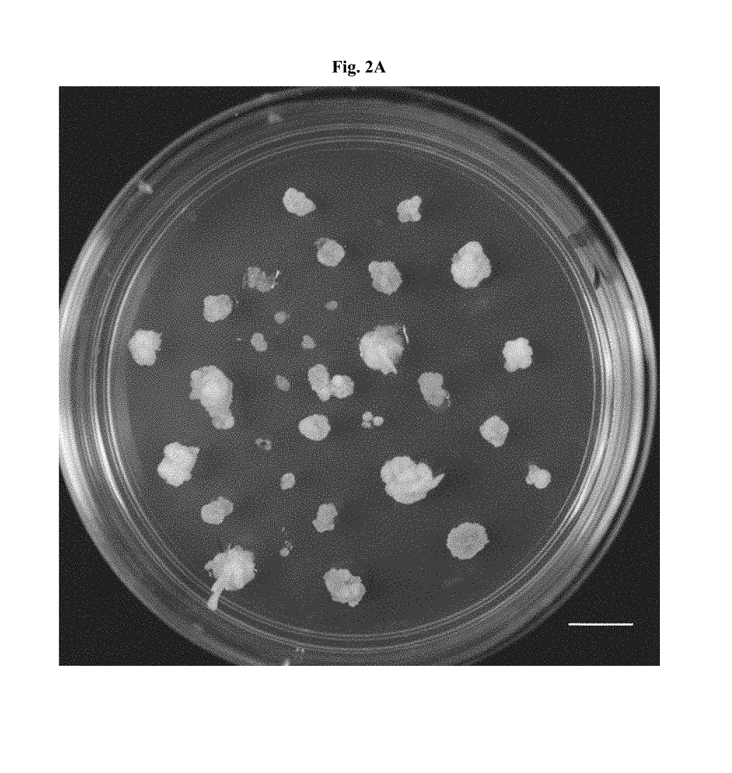

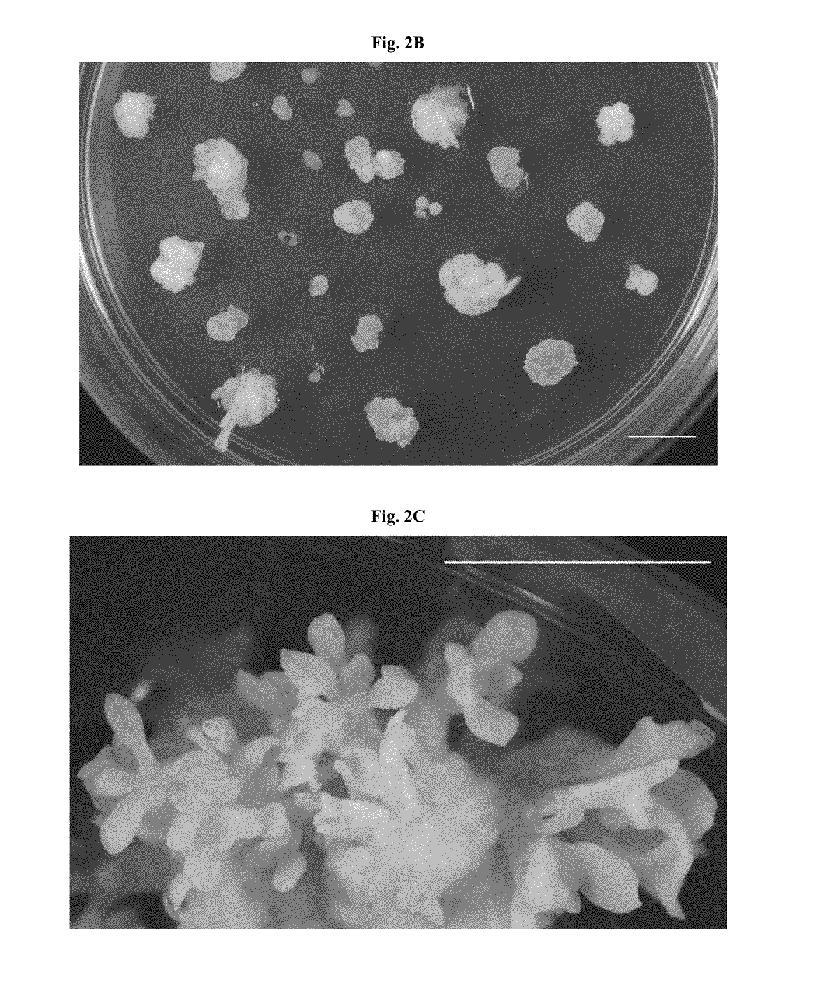

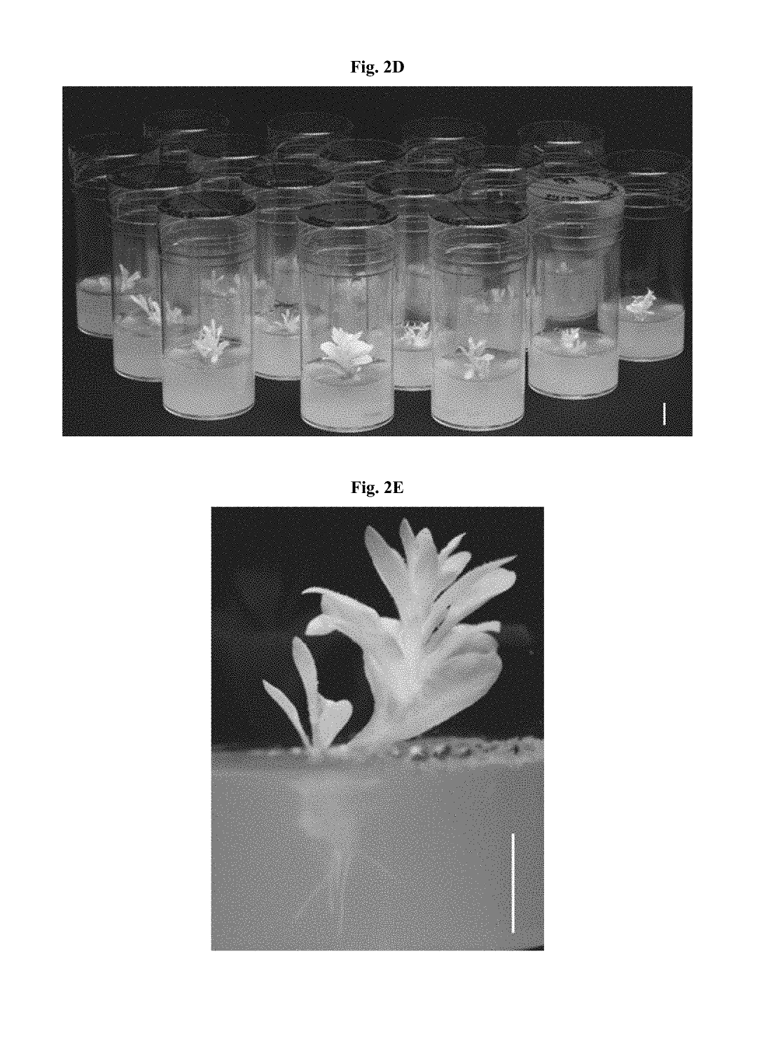

FIG. 2A depicts colonies formed from tobacco protoplasts 50 days after transfection with mRNAs of Cas9, NPTII and PDS gRNA. Scale bar=1 cm.

FIG. 2B depicts colonies formed from tobacco protoplasts 50 days after transfection with mRNAs of Cas9, NPTII and PDS gRNA. Scale bar=1 cm.

FIG. 2C depicts pds mutant shoots from colonies formed from tobacco protoplasts 60 days after transfection with mRNAs of Cas9, NPTII and PDS gRNA. Scale bar=1 cm.

FIG. 2D depicts pds mutant shoots from colonies formed from tobacco protoplasts 90 days after transfection with mRNAs of Cas9, NPTII and PDS gRNA. Scale bar=1 cm.

FIG. 2E depicts pds mutant plantlets in rooting medium from colonies formed from tobacco protoplasts 105 days after transfection with mRNAs of Cas9, NPTII and PDS gRNA. Scale bar=1 cm

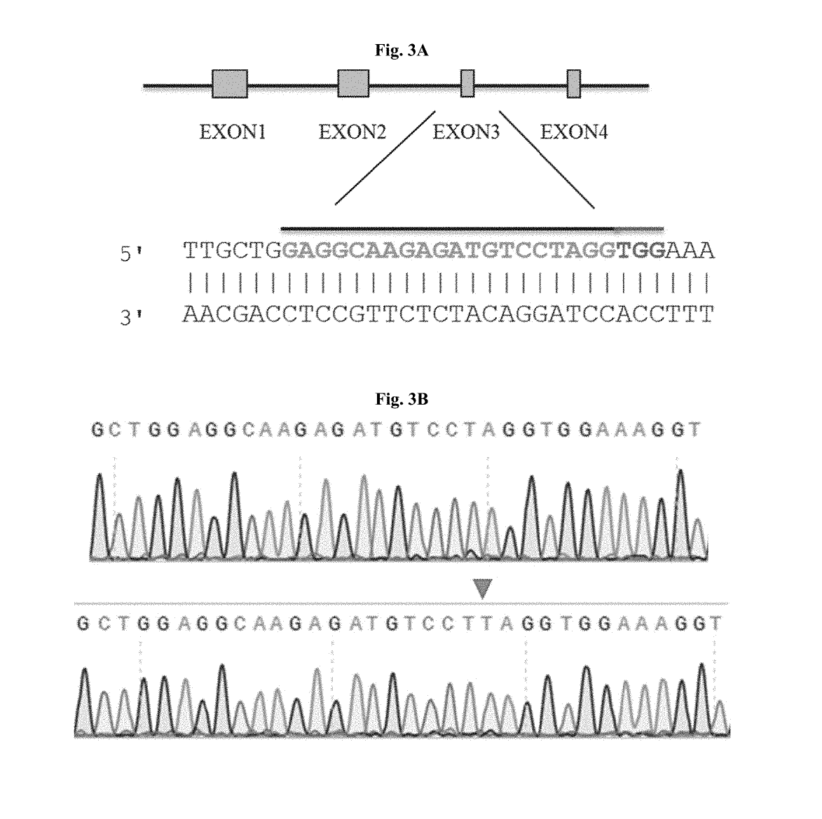

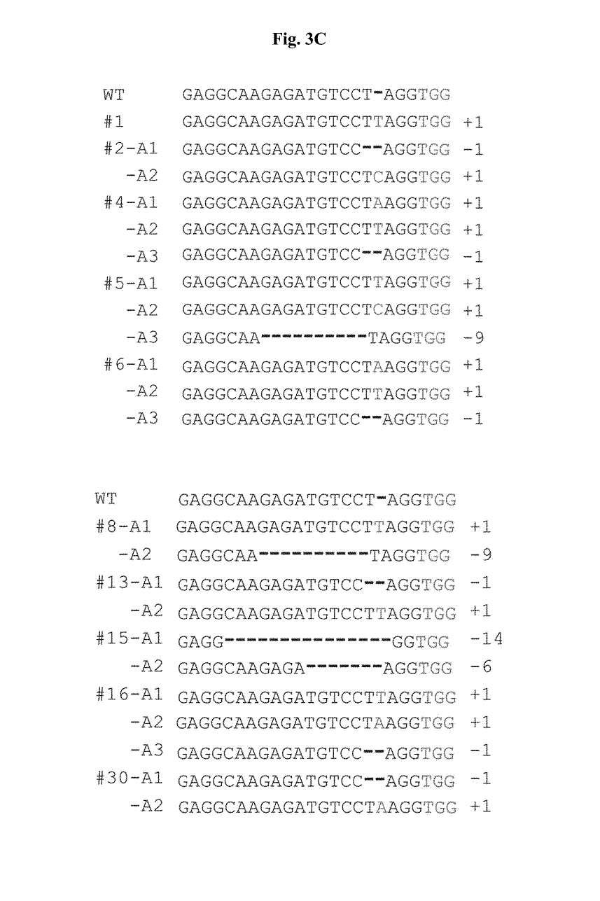

FIG. 3A depicts the target sequence in the NtPDS gene. The PAM sequence is shown in red and the target sequence is shown in green. FIG. 3A includes SEQ ID NOs 9-10, respectively, in order of appearance.

FIG. 3B provides the DNA sequences of the wild type (upper) (SEQ ID NO: 11) and a pds mutant (lower) (SEQ ID NO: 12). The blue triangle indicates an inserted nucleotide.

FIG. 3C provides the DNA sequences of the wild-type (WT) and pds mutants (SEQ ID NOS 13-37, top to bottom, respectively, in order of appearance). The PAM sequence is shown in red, insertions are shown in blue and deletions are indicated by dashes.

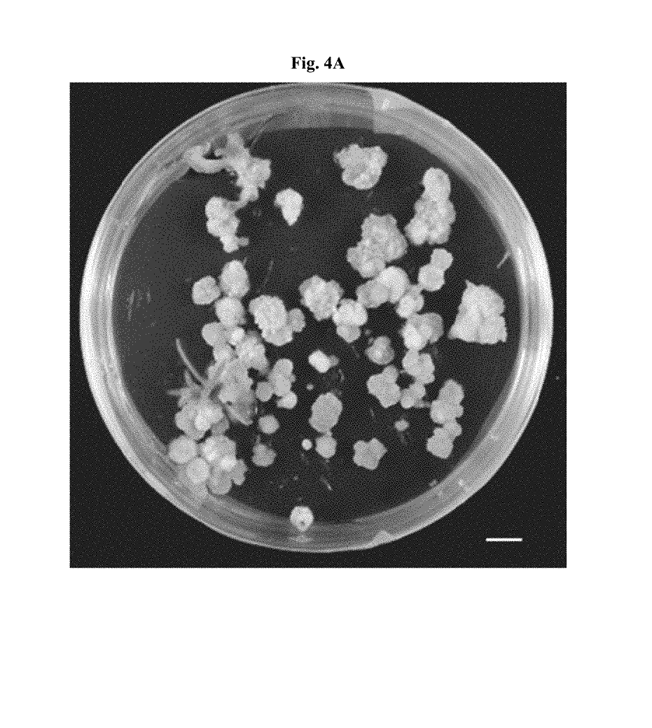

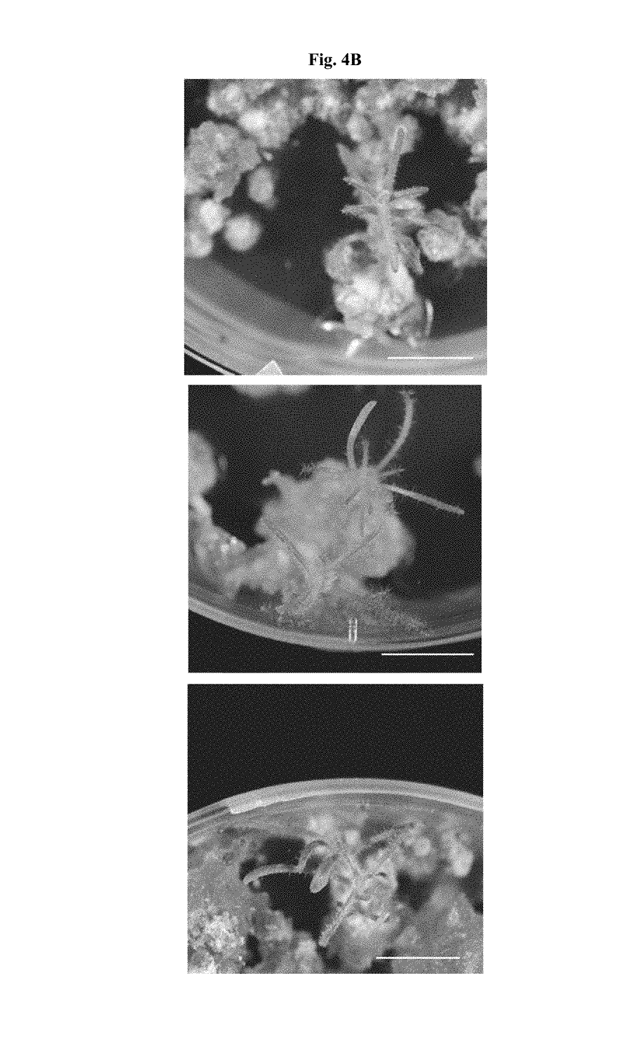

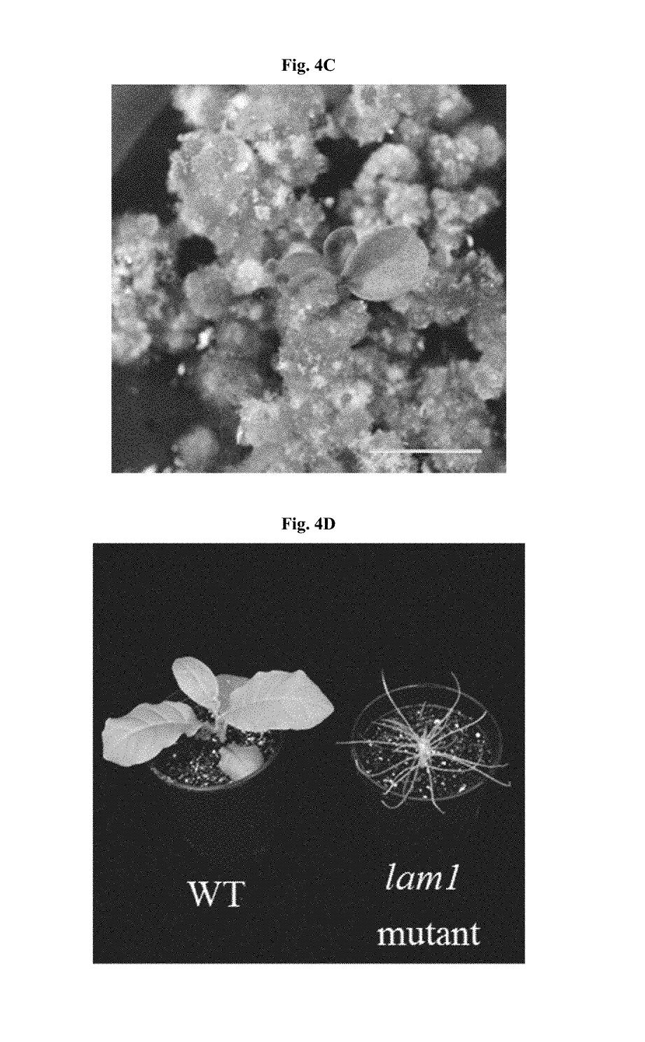

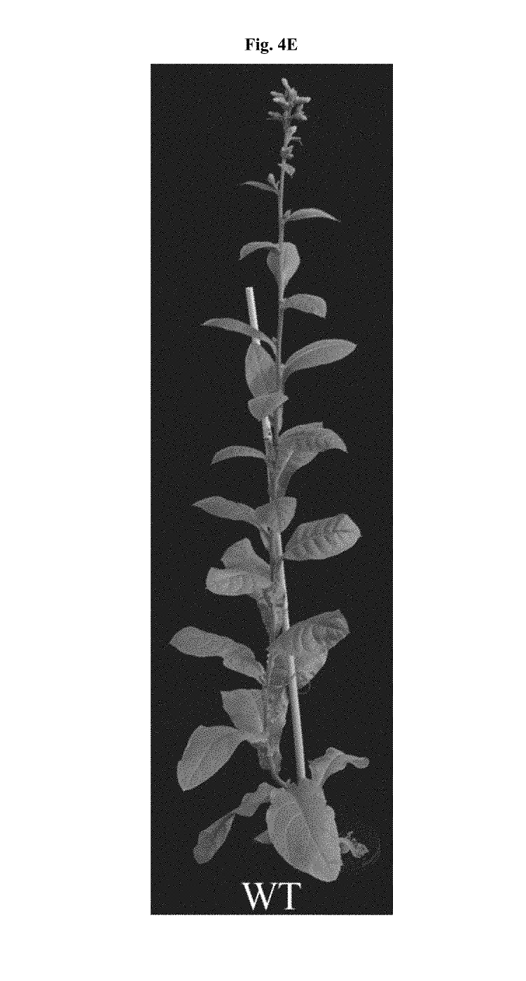

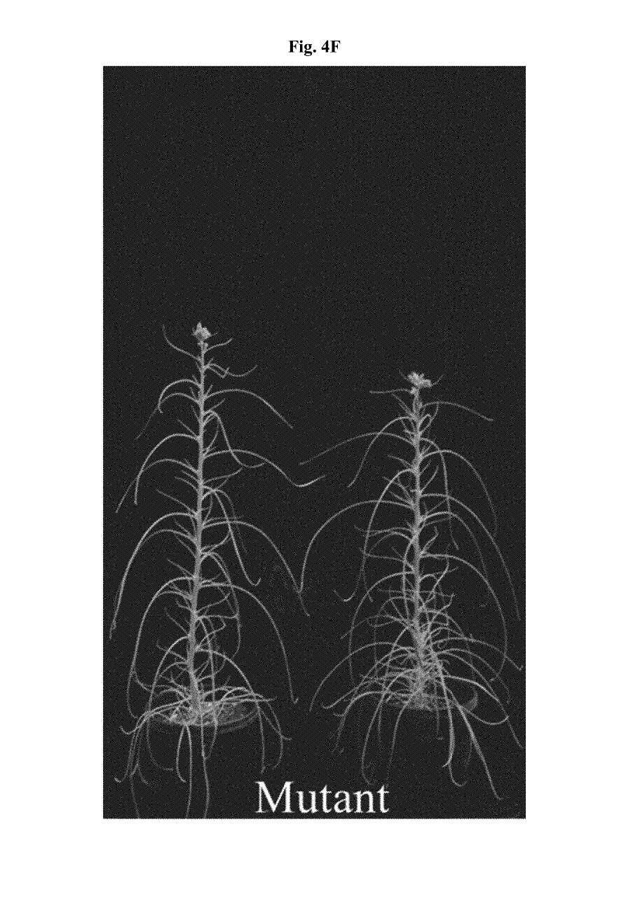

FIG. 4A depicts colonies formed from tobacco protoplasts 50 days after transfection with mRNAs of Cas9, NPTII and LAM1 gRNA. Scale bar=1 cm.

FIG. 4B depicts regeneration of lam1 mutant shoots 60 days after transfection. Scale bar=1 cm.

FIG. 4C depicts regeneration of control WT shoots 60 days after transfection with mRNAs of Cas9 and LAM1 gRNA. Scale bar=1 cm.

FIG. 4D depicts wild-type and lam1 mutant plants in soil 110 days after transfection.

FIG. 4E depicts a wild-type plant grown in a greenhouse.

FIG. 4F depicts lam1 mutant plants grown in a greenhouse.

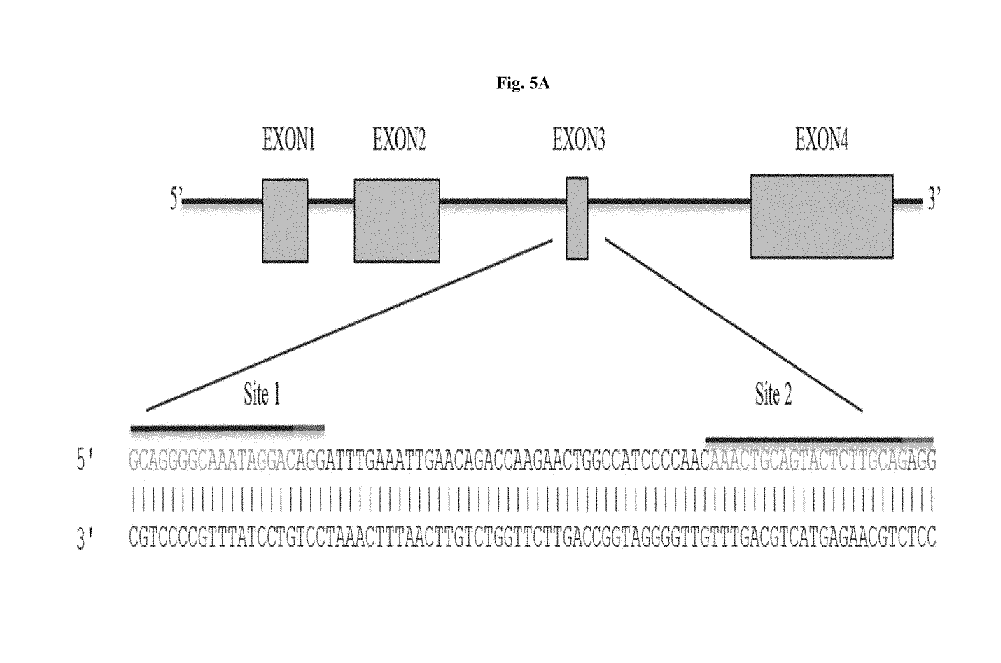

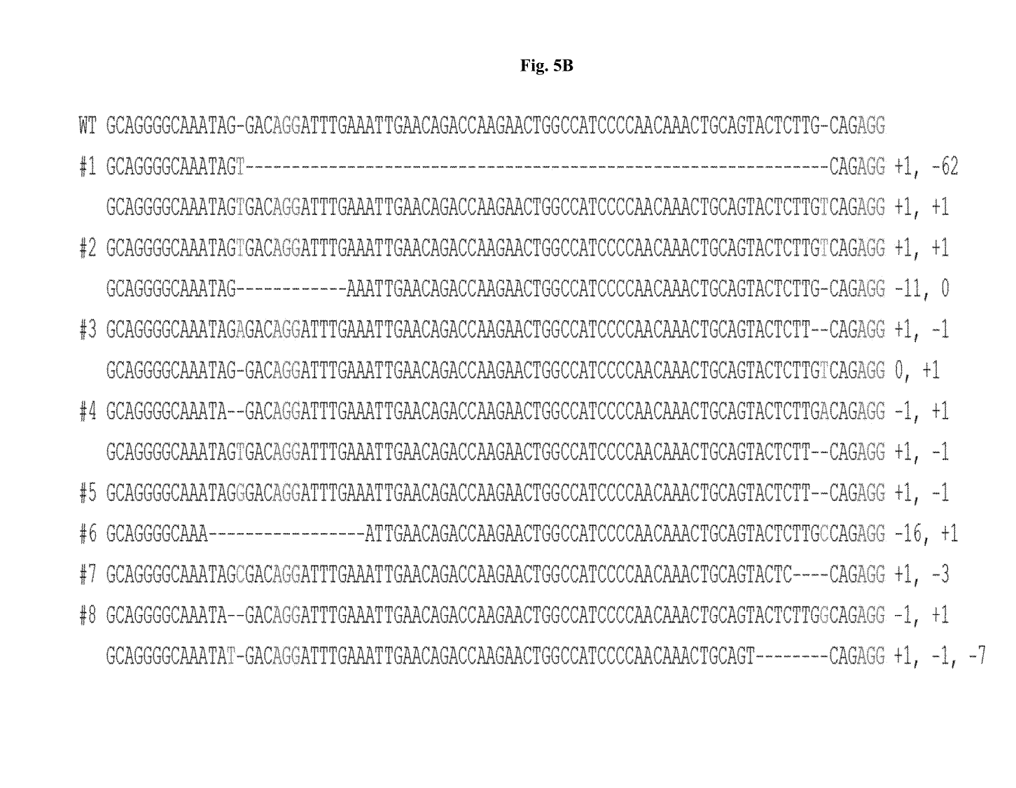

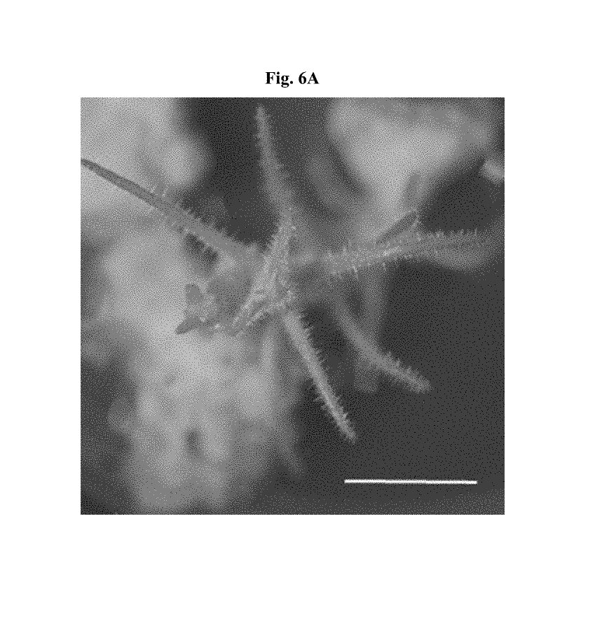

FIG. 5A depicts the target sequences (Site 1 and Site 2) in the NtLAM1 gene. The PAM sequences are shown in red and the target sequences are shown in green.

FIG. 5B provides the DNA sequences of the wild-type (WT) and lam1 mutants for Sites 1 and 2 (SEQ ID NOS 38-53, respectively, in order of appearance). The PAM sequence is shown in red, insertions are shown in blue and deletions are indicated by dashes.

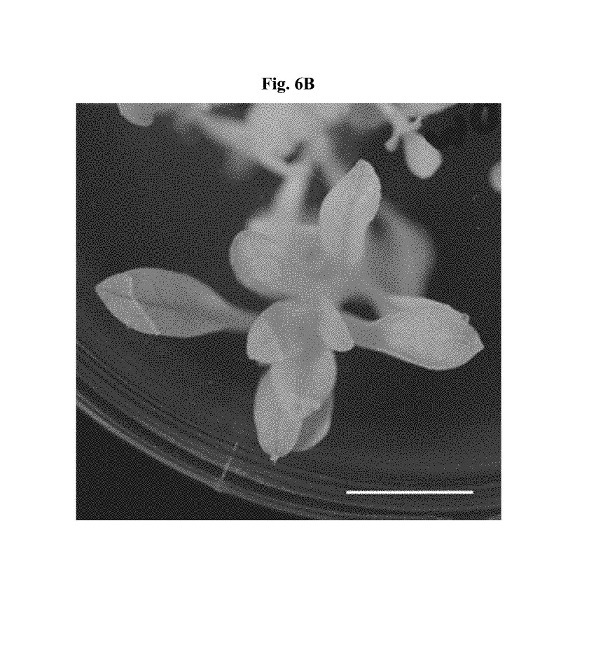

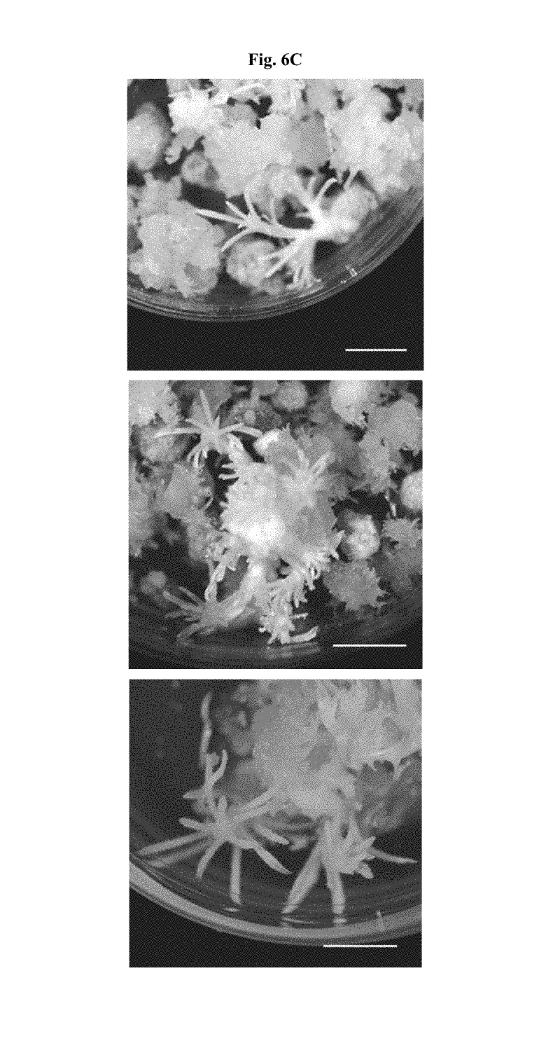

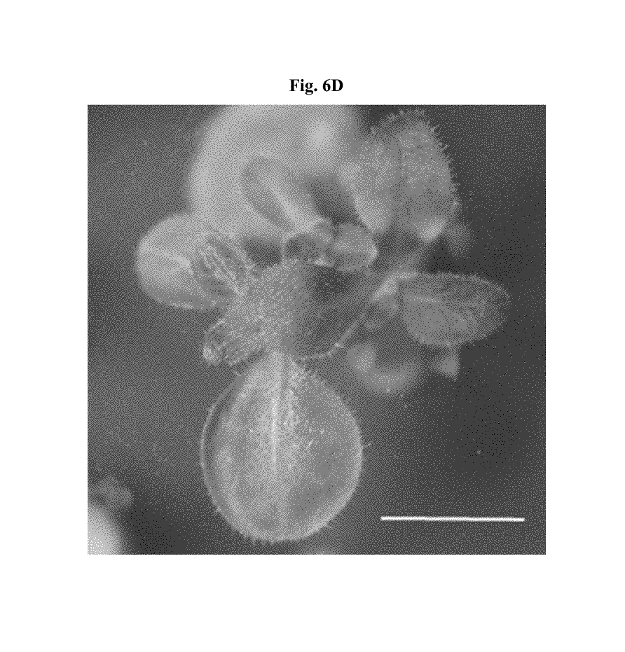

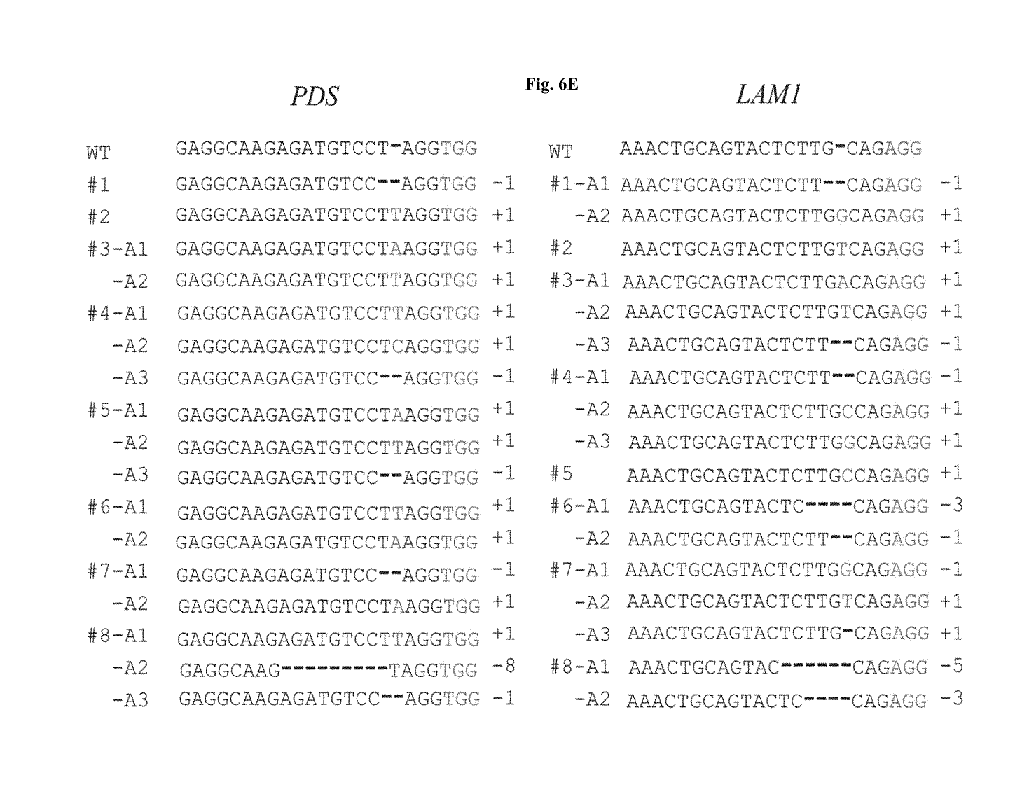

FIG. 6A depicts regeneration of lam1 shoots 60 days after transfection with mRNAs of Cas9, NPTII, PDS gRNA and LAM1 gRNA. Scale bar=1 cm.

FIG. 6B depicts regeneration odpds mutant shoots 60 days after transfection with mRNAs of Cas9, NPTII, PDS gRNA and LAM1 gRNA. Scale bar=1 cm.

FIG. 6C depicts regeneration of pds/lam1 double mutant shoots 60 days after transfection with mRNAs of Cas9, NPTII, PDS gRNA and LAM1 gRNA. Scale bar=1 cm.

FIG. 6D depicts regeneration of wild-type shoots 60 days after transfection without mRNAs of Cas9, NPTII, PDS gRNA and LAM1 gRNA and without selection. Scale bar=1 cm.

FIG. 6E provides the DNA sequences of the wild-type (WT) and lam1 and pds double mutants. The PAM sequence is shown in red, insertions are shown in blue and deletions are indicated by dashes. FIG. 6E discloses SEQ ID NOS 54-89, top to bottom, left to right, respectively, in order of appearance.

While the present disclosure is susceptible to various modifications and alternative forms, specific embodiments have been shown in the figures and are herein described in more detail. It should be understood, however, that the description of specific embodiments is not intended to limit the invention to the particular forms disclosed, but on the contrary, this disclosure is to cover all modifications and equivalents as illustrated, in part, by the appended claims.

### DESCRIPTION

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The use of the term “or” in the claims is used to mean “and/or” unless explicitly indicated to refer to alternatives only or the alternatives are mutually exclusive.

Unless otherwise defined, all technical and scientific terms used herein have the same meaning as commonly understood by one of ordinary skill in the art.

As used herein, the term “proliferate” or “proliferation” should be understood to mean grow or multiply in number. “Resistance” to a selection agent or selection condition means the cell's ability to continue to proliferate in the presence of the selection agent or selection condition at or near a rate that would be expected by one of ordinary skill in the art in a cell under standard growth conditions in the absence of the selection agent or selection condition. Alternatively, when negative selection processes are used, selection is based on those cells which demonstrate reduced proliferation as compared to the control cells that were not transfected with the RNA molecule. Where the RNA molecule encodes a visual reporter protein, selection is based simply on the visual appearance of the cell and a selection agent is generally unnecessary in this instance.

The present disclosure provides description to the accompanying drawings, in which some, but not all embodiments of the subject matter of the disclosure are shown. Indeed, the subject matter may be embodied in many different forms and should not be construed as limited to the embodiments set forth herein, rather, these embodiments are provided so that this disclosure satisfies all the legal requirements.

All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The present disclosure is related to methods, kits, and compositions to permit selection of cells and/or gene editing in cells and/or selection of edited cells using RNA. In some embodiments, a plurality of cells is exposed to a first RNA molecule under conditions sufficient to promote transfection of the first RNA molecule into one or more of the plurality of cells, the first RNA molecule and/or a peptide or protein encoded by the first RNA molecule permits selection of the one of more of the plurality of cells transfected with the first RNA molecule based on a first selectable property. By way of example, but not limitation, selectable properties conferred by a RNA molecule and/or a peptide or protein encoded by the RNA molecule include restance to a selection agent, a visually detectable difference such as a color change, growth under starvation conditions, impaired growth due to production of a toxic agent, impaired growth due to conversion of a non-toxic agent to a toxic agent. Resistance to a selection agent can be achieved by, for example, transfecting cells with a RNA encoding a peptide or protein that confers resistance to the selection agent or by transfecting the cells with a silencing RNA which inhibits or reduces the expression of a gene which confers susceptibility to a selection agent. In one embodiment, a method is provided for selecting for cells resistant to a selection agent by conferring such resistance through transfection of an RNA molecule into the cell. The RNA molecule encodes a protein or peptide that acts to deactivate or reduce the activity of a compound (selection agent) that kills cells or reduces the cells' ability to proliferate or regenerate. In one instance, the protein or peptide encoded by the transfected RNA molecule may act directly on the selection agent to reduce, inhibit or deactivate its activity in the cell. In other instances, the protein or peptide encoded by the transfected RNA molecule may act to inhibit downstream targets of the selection agent or to reduce expression of other factors in the cell necessary for the selection agent to elicit a cellular response. It should be understood that cells being transfected with the RNA molecule may also comprise an endogenous RNA molecule having a similar sequence to the transfected RNA molecule, but where the endogenous RNA molecule is not expressed at a high enough level to confer resistance to the selection agent.

Examples of RNA molecules used for providing resistance to selection agents include, but are not limited to, nptII (SEQ ID NO: 1), hph (SEQ ID NO: 2) (which can be used to confer resistance to, for example, hygromycin), bar (SEQ ID NO: 3) (from Streptomyces hygroscopicus for use with phosphinothricin (PPT) herbicides) and epsps (SEQ ID NO: 4) (confers resistance to glyphosphate). For example, NPTII RNA codes for the aminoglycoside 3′-phosphotransferase (denoted aph(3′)-II or NPTII) enzyme, which inactivates a range of aminoglycoside antibiotics such as kanamycin by phosphorylation. Other plant derived marker genes useful as RNA molecules in the present methods for conferring resistance to antibiotic-based or herbicide-based selection agents or other selection conditions includes, but is not limited to, the following: Arabidopsis thaliana ATP binding cassette (ABC) transporter (At-WBC19) (for kanamycin in tobacco, hybrid aspen, alfalfa, rye, oat, wheat, triticale, hairy vetch, common vetch, white clover, red clover, radish, cotton, sugarcane, sugarbeet, tall fescue, ryegrass, switchgrass, sorghum, peanut, sunflower, canola, or muskmelon); A. thaliana DEF2 (peptide deformylase) and GPT (UDP-N-acetylglucosamine:dolichol phosphate Nacetylglucosamine-1-P transferase) (for actinonin and tunicamycin in tobacco and Arabidopsis); glyphosphate oxydoreductase (gox) gene (for use with glyphosphate); gat4061 and gat4621 genes (encoding GAT proteins for use with glyphosphate herbicide); pat gene (from S. viridochromogenes for use with PPT herbicide); galT gene encoding UDP-glucose/galactose-1-phosphate uridyltransferase; mutant genes encoding acetolactate/acetohydroxyacid synthase for use with sulfonylurea or imidazolinone herbicides); cyanamide hydratase (cah) gene (from Myrothecium verrucaria for use with cyanamide; GSA gene encoding Glutamate 1-semialdehyde aminotransferase (for use with L-glutamate-1-semialdehyde); dehl gene (from Pseudomonas putida coding for a dehalogenase for use with dalapon); organophosphorus hydrolase (oph) gene (from Pseudomonas diminuta for use with organophosphorus pesticides); DOG^{R}1 gene encoding 2-deoxyglucose-6-phosphate phosphatase (for use in tobacco and potato with 2-deoxyglucose); human P450 genes (for use with herbicides in Arabidopsis, tobacco, potato and rice); mutated versions the phytoene desaturase (pds) gene (for use with fluridone, norflurazon and flurtamone) mutant or native forms of the protoporphyrinogen oxidase (ppo) gene (for use with Arabidopsis, maize and rice with butafenacil); and mutant alpha-tubulin Tub1 gene (from goose grass for use with herbicides of the dinitroaniline and phosphoroamidate families in finger millet, soybean, flax, tobacco and barley). By way of example but not limitation, other plant varieties that can be used include alfalfa, rye, oat, wheat, triticale, hairy vetch, common vetch, white clover, red clover, radish, cotton, sugarcane, sugarbeet, tall fescue, ryegrass, switchgrass, sorghum, peanut, sunflower, and canola.

In some instances, the RNA molecule is a silencing RNA (siRNA). Here, the siRNA molecule reduces or inhibits the expression of an endogenous gene that confers a cell's susceptibility to the selection agent. One specific example is a siRNA specific to the MAR1 gene. Reducing expression of MAR1 confers resistance to kanamycin in A. thaliana. In addition to siRNA, the RNA molecule can be other forms of RNA that are known to interfere with or reduce gene expression.

It should be understood that the selection process of the present disclosure could be practiced with multiple RNA molecules encoding different peptides or proteins to confer resistance to different selection agents. Moreover, it should be understood that the selection process described herein is not limited to use in connection with gene editing, but could be used in connection with any process in which cell transfection methods are required to promote entry of material into a cell.

Transfection of the RNA molecule (and any other nucleic acids) can be achieved by standard methods for the relevant cell types employed and will be apparent to one of skill in the art upon selection of the cell type and RNA molecule. For example, transfection in a plant cell may be achieved via exposure to a solution of polyethylene glycol (discussed in more detail in the Examples below), electroporation, particle bombardment, or using standard transfection reagents suitable for the particular cell type being transfected.

Electroporation can be performed according to methods known in the art and with some modifications as described below. After washing freshly isolated protoplasts free of the enzymes used for the digestion of the cell wall, cells in the pellets are resuspended in 10 ml electroporation buffer (Sucrose 0.4 M (13.7%), HEPES 2.4 g/L, KCl 6 g/L, CaCl₂.2H₂O 600 mg/L, pH 7.2 (with KOH)). Next, the protoplast concentration is determined using a hemocytometer, and 1×10⁶ protoplasts are aliquoted into 15-ml conical centrifuge tubes, and the protoplasts are spun down (50 g for 5 min). After removing the supernatant, each protoplast samples is gently resuspend in 0.5 ml electroporation buffer with 10-100 μg/ml of the desired RNA using a disposable plastic transfer pipet. The protoplast samples are transferred with a disposable plastic transfer pipet to electroporation cuvettes and let stand at RT for 5 min. In the next step, the protoplasts are resuspended by shaking the electroporation chamber gently, and then a single electric pulse (130 V and 1,000 μF) is immediately applied. After the electroporation, the cuvettes are incubated at room temperature for 20-30 min without moving. The protoplasts are then resuspended by gentle agitation of the electroporation chamber and transferred to a conical centrifuge containing 5 ml of W5 solution (154 mM NaCl, 125 mM CaCl₂, 5 mM KCL, 5 mM glucose; pH 5.8). The electroporation chamber is rinsed with 0.5 ml W5 and then the rinse is combined with the protoplast sample. When all the protoplast samples have been electroporated, the protoplasts are pelleted and then embedded to be cultured as described in the examples below for the PEG method.

Particle bombardment can be performed by methods known in the art. In one particular example, 10 μg Cas9 mRNA is premixed with gRNA along with equal molar concentrations of selection RNA and co-coated on gold particles (0.6 μm, 1.5 mg) by adding 1/10 volume of 5 M ammonium acetate and 2 volumes of 2-propanol sequentially as recommended by the manufacturer (Bio-Rad Laboratories, Richmond, Calif.). After continual vortexing for 5-10 min, the mixture can be placed at −20° C. for 1 h or longer. After a quick centrifugation (2,000 g for 3 s), the supernatant can be discarded and the RNA-microcarrier complexes are washed with 100% ethanol twice and resuspended in 40 μl 100% ethanol. The suspension is mixed gently and 10 μl aliquots are placed onto a macrocarrier at a time for bombardment. The helium pressure and shooting distance can be 1100 psi and 6 cm.

Upon transfection of the cells with the exogenous RNA molecules, the cells are exposed to a selection agent. The selection agent may include antibiotics or herbicides (as disclosed in the list of exemplary RNA molecules above), but can also include a host of other materials, that when administered or applied to cells transfected with the RNA molecules, permit selection of the RNA transfected cells based on the cells displaying resistance to the selection agent. However, other types of selection agents and procedures can be used as discussed below.

The duration of the exposure to the selection agent will vary based on agent being used, the type of cell, and whether the selection process is positive or negative. In instances where the selection agent is an antibiotic such as kanamycin, and the cell is a plant cell such as a tobacco protoplast, and the RNA molecule is, for example, ntpII, the exposure may range from 1 day to 10 days, from 5 days to 7 days, and more preferably 6 days. In some embodiments, the plurality of cells can be exposed to a selection agent for between 1 day to 45 days, 1 day to 30 days, 1 day to 10 days, 5 days to 7 days, or 6 days.

In some instances, the selection agent can include a passive mechanism such as starvation wherein the cells are transfected with an RNA molecule encoding a protein or peptide that allow the cells to survive on carbohydrates that cannot otherwise be metabolized. One example of an RNA molecule used with this type of passive selection agent is the manA gene which encodes an E. coli-derived phosphomannose isomerase (PMI). Thus, as used herein, a “starvation condition” should be understood to mean a condition in which normal, wild-type cells are unable to survive or proliferate.

In other instances, the selection process can occur without a selection agent through negative selection wherein the cell is transfected with an RNA molecule that encodes a protein or peptide that impairs cellular growth due to intracellular production of a toxic agent. Additionally, the RNA molecule can be a reporter gene that permits visual selection, such as the mutant allele of the apple MYB10 gene.

In other instances, the selection agent can be a non-toxic agent. Here, the RNA molecule encodes a protein or peptide that converts the non-toxic agent into a toxic agent. For example, the bacterial haloalkane dehalogenase (dhla) gene converts non-toxic substances into chlorinated alcohol and chlorinated aldehyde. Another example is the E. Coli codA gene which could be used with 5-fluorocytosine (non-toxic) as the selection agent. The codA gene product converts the 5-fluorocytosine into 5-fluorouracil which is cytotoxic.

In certain embodiments, the selection methods described above can be used to select cells transfected with additional nucleic acids for performing gene editing functions. In certain embodiments, the additional nucleic acids may comprise RNA or DNA. For example, the additional nucleic acid (in addition to the first RNA molecule used for selection purposes) may be a DNA plasmid construct for stable integration into a cell's genome. In other instances, the additional nucleic acids may be additional RNA molecules for performing gene editing functions. Specifically, a second RNA molecule encoding the Cas9 protein and a third RNA molecule providing a guide RNA may be utilized in the present methods. The guide RNA (gRNA) comprises a scaffold sequence for Cas9 binding and a target sequence, wherein the target sequence defines the genomic region of the plurality of cells to be edited. SEQ ID NO: 91 discloses a gRNA sequence containing a 20 nucleotide target sequence 5′ adjacent to a Cas9 scaffold sequence and 3′ terminal poly-U sequence. The genomic region can vary depending on the desired phenotype. For example, the genomic region may include the phytoene desaturase (PDS) gene or LAM1 gene in tobacco protoplasts. In some embodiments, the editing of the genomic region results in a selectable phenotype. Such selectable phenotypes can include, by way of example but not limitation, changes in growth characteristics and other visual changes. The examples below provide further description regarding DNA-free methods to utilize the CRISPR gene editing system.

The molar ratio of Cas9 mRNA to gRNA may be from about 1:1 to about 1:100, from about 1:1 to 1:50, from about 1:1 to about 1:40, from about 1:1 to about 1:30, from about 1:1 to 1:20, from about 1:1 to about 1:10, from about 1:2 to about 1:90, from about 1:3 to about 1:80, from about 1:4 to about 1:70, from about 1:5 to 1:60, from about 1:6 to about 1:50, from about 1:7 to about 1:40, from about 1:8 to about 1:30, from about 1:10 to about 1:20, from about 1:10 to about 1:30, from about 1:10 to about 1:40, from about 1:10 to about 1:50, and any intermediate ranges between the foregoing. In certain embodiments, the molar ratio of Cas9 mRNA to gRNA is 1:20. It should be understood that the particular ratio used may depend on the type of cells being transfected and the number of different gRNAs that are used in a single transfection.

The present methods may further comprise subjecting the selected transfected cells to conditions sufficient to promote growth of the cells into a plant. One of ordinary skill in the art would understand which conditions are appropriate for a given cell type and further description of such growth conditions are described in further detail in the examples below. The present methods may also further comprise harvesting a seed from the plant.

The present disclosure also provides kits for selection of transfected cells. In one embodiment, the kit comprises at least a first RNA molecule and a selection agent of the types described herein above along with a transfection reagent. The transfection reagent may comprise any materials needed to perform a transfection method as described herein or which would otherwise be apparent to one of ordinary skill in the art. The kit may further comprise additional nucleic acids such as RNA encoding Cas9 and associated gRNA. It should be understood that the kits of the present disclosure may comprise any of the components used in connection with the methods described herein.

The present disclosure further provides for genetically-modified plants produced by the methods described herein. Traditionally, genetically-modified plants require the use of foreign DNA to perform the selection and/or gene editing function. Here, we provide for the first time a method to select for and perform gene editing without the use of foreign DNA and thus provide plants and seeds with mutations induced without foreign DNA integrated into the genome.

The following examples are included to demonstrate preferred embodiments of the present disclosure. Those of skill in the art should, in light of the present disclosure, appreciate that many changes can be made in the specific embodiments which are disclosed and still obtain a like or similar result without departing from the concept, spirit and scope of the present disclosure.

### EXAMPLES

The following examples demonstrate specific embodiments employing the methods of the present disclosure to produce mutant plants without the integration of foreign DNA into the genome.

The following materials and methods were used in the Examples as applicable.

mRNA and gRNA Synthesis.

hCas9 gene or nptII (kanamycin resistance) gene were amplified from pRGEB31, and then cloned into a pENTR/D-TOPO vector to generate pENTR-Cas9 vector or pENTR-nptII vector, respectively. A T3 promoter was added upstream of the hCas9 gene or nptII gene open reading frame. A SwaI restriction enzyme site was added downstream of the hCas9 gene or nptII gene open reading frame to linearize the plasmid. Cas9 mRNA or nptII was produced by in vitro transcription from a linearized pENTR-Cas9 vector or pENTR-nptII vector, respectively, using mMESSAGE mMACHINE T3 kit and the Poly (A) Tailing kit (Ambion). In vitro transcriptions were carried out at 37° C. for 2 h in a total volume of 20 μl with 1 μg purified linear DNA template. To remove template DNA, 1 μl of TURBO DNase was added, mixed well, and incubated at 37° C. for 30 min, followed by extraction with phenol-chloroform.

Templates for guide RNA transcription were generated by oligo-extension using no template PCR with Phusion polymerase. In detail, two overlapping primers, one containing T7 promoter and 20 (or 17 in the case of LAM1 Site 2 discussed below (SEQ ID NO: 98)) nucleotides of Cas9 target sequence (5′-primer GCGGCCTCTAATACGACTCACTATAGG NNNNNNNNNNNNNNNNNNNNGTTTTAGAGCTAGAAATAGCA (SEQ ID NO: 5)), and second containing sgRNA backbone (common reverse primer, universal sgR1, 5′-AAAAAAGCACCGACTCGGTGCCACTTTTTCAAGTTGATAACGGACTAGCCTTATTTT AACTTGCTATTTCTAGCTCTAAAAC (SEQ ID NO: 6)) were combined in a PCR. PCR was performed under the following conditions: 98° C. for 2 min, followed by 3 cycles of 98° C., 10 sec; 53° C., 20 sec; 72° C., 20 sec; and 72° C. for 2 min. Then one primer containing T7 promoter and 20 (or 17 in the case of LAM1 Site 2 discussed below (SEQ ID NO: 99)) nucleotides of Cas9 target sequence (5′-primer GCGGCCTCTAATACGACTCACTATAGG NNNNNNNNNNNNNNNNNNNNGTTTTAGAGCTAGAAATAGCA (SEQ ID NO: 7)) and a backbone reverse (Universal sgR2, 5′-AAAAAAGCACCGACTCGGTGC (SEQ ID NO: 8)) primer were added to the first round PCR mixture, and second round PCR was performed under the following conditions: 98° C. for 2 min, followed by 30 cycles of 98° C., 10 sec; 58° C., 20 sec; 72° C., 20 sec; and finally 72° C., 2 min.

PCR product was purified by using the Wizard® SV Gel and PCR Clean-Up System (Promega), and the DNA was used as a template for an in vitro sgRNA synthesis with the T7 shortscript kit (Ambion). 1 μg purified DNA template was used and incubated for 4 h. The reaction mixture was treated with TURBO DNase provided in the kit. The RNA was purified by phenol chloroform extraction and alcohol precipitation. 0.5 μl sgRNAs were run on a gel to confirm integrity before transfection and quantified by using a Nanodrop spectrophotometer.

SSA Recombination Assay.

A single-strand annealing (SSA) reporter was generated by disrupting the eGFP gene by duplicating an internal 141 bp region and separating the duplicated region with a 17 bp fragment containing XhoI and BglII restriction enzyme sites. SSA reporter plasmid was digested with XhoI and BglII restriction enzymes, and the sequence containing one target site was inserted into it. The OsU3 promoter and the HPH gene in pRGEB31 were replaced with AtU6 promoter and the Bar gene, respectively, referred to as pRGEB31-AtU6-PPT. pRGEB31-AtU6-PPT was digested with BstBI and PmeI to remove the Cas9 gene, and was named pRGEB31-AtU6-PPT-Cas9-gRNA that target the sequence inserted in SSA reporter plasmid was ligated into pRGEB31-AtU6-PPT-Cas9, the resulting plasmid is called pRGEB31-AtU6-PPT-Cas9-gRNA. The SSA reporter, pRGEB31-AtU6-PPT, pRGEB31-AtU6-PPT-Cas9-gRNA plasmid or Cas9 mRNA and gRNA were transfected by PEG into protoplasts. After an additional 48 h, cells that were positive and those that were negative for eGFP fluorescence were scored in eight regions, and the percentage of positive cells relative to all viable cells was determined.

Protoplast culture. Protoplasts were isolated as previously described from Arabidopsis, tobacco, tomato, rice and Medicago with minor modifications. Seeds of Arabidopsis (Arabidopsis thaliana) ecotype Wassilewskija (WS) and tobacco (Nicotiana tabacum) cv Xanthi were sterilized in 70% ethanol for 2 min, washed at least five times in distilled water, dried overnight in hood, and then keep sterile for later use. Seeds of rice (Oryza sativa L.) cv. Nipponbare and tomato (micro tom) were sterilized in a 70% ethanol for 1 min, 2.5% sodium hypochlorite for 30 min, washed at least five times in distilled water, dried overnight in hood, and then keep sterile for later use. Tobacco and rice seeds were incubated on MS medium (Murashige and Skoog solid medium) (Phyto tech) supplemented with 2% sucrose under a photoperiod of 16 h light (about 150 μmol m⁻² s⁻¹) and 8 h dark at 26° C. for 28 days and 7-10 days, respectively. Tomato seeds were placed on double layer of pre-sterilized filter paper in a glass Petri dish, 5 ml sterile distilled water was added and the dish was placed in a dark incubator at 28° C. After 3-5 days, a portion (2-5 mm) of the hypocotyl was transferred into a plant container with MS medium supplemented with 2% sucrose under a photoperiod of 16 h light (about 150 μmol m⁻² s⁻¹) and 8 h dark at 26° C. Arabidopsis was incubated on MS medium supplemented with 1% sucrose, and then were transferred to the growth chamber under a photoperiod of 12 h light (about 50 μmol m⁻² s⁻¹) and 12 h dark at 22° C. after 2 days at 4° C. in dark conditions. After 3 days at 4° C. in dark conditions, Medicago (Medicago truncatula) were transferred to a growth chamber under 12-h light photoperiod with a photon flux density of 40-60 μmol m⁻² s⁻¹, 80% relative humidity, and temperature of 22° C. before protoplast isolation was initiated.

For protoplast isolation, the leaves of 17-20 d Arabidopsis seedlings were digested with enzyme solution (0.1% (wt/vol) cellulase R10, 0.03% (wt/vol) macerozyme R10, 0.04% (wt/vol) Driselase, MGG) overnight in the dark at 24° C. The stem and sheath of 10 d rice seedlings were digested with enzyme solution (1.5% (wt/vol) cellulase R10, 0.5% (wt/vol) macerozyme R10, 0.6 M mannitol, 10 mM KCl, 20 mM MES [pH 5.7]) in the dark for 5-6 h with gentle shaking (70 rpm) at 24° C. The mixture was filtered with 70 μm mesh and then washed with two volume of W5 solution. Three or four week old tomato were digested with enzyme solution (1.5% (wt/vol) cellulase R10, 0.3% (wt/vol) macerozyme R10, TSE2 (see supplemental data)) for 8 h in the dark at 29° C. The mixture was filtered with 40 μm mesh and centrifuged to float the protoplasts. In the case of tobacco protoplasts, 4-week-old Nicotiana tabacum leaves were digested with enzyme solution (1.3% (wt/vol) cellulase R10, 0.7% (wt/vol) macerozyme R10, K4 (see supplemental data)) for 15-18 h at 25° C. in the dark. Subsequently, the mixture was filtered with 100 μm mesh before protoplasts were collected by centrifugation at 80 g in a round-bottomed tube for 5 min. To obtain intact protoplasts, protoplasts were floated twice with K4 medium and wash with an equal volume of W5 solution once. The intact protoplasts were re-suspended in 0.5 ml W5 solution and stabilized for 30 min at 4° C. before PEG-mediated transfection. Finally, protoplasts were re-suspended in MMG solution and counted under the microscope using a hemocytometer. Protoplasts were diluted to a density of 5×10⁵ protoplasts/ml of MMG solution (Arabidopsis and rice, 0.4 M mannitol, 15 mM MgCl₂, 4 mM MES [pH 5.7], tomato, Medicago and tobacco, 0.5 M mannitol, 15 mM MgCl₂, 4 mM MES [pH 5.8]).

Protoplast Transfection.

PEG-mediated RNA transfections were performed as previously described. Briefly, 5×10⁴ protoplast cells were transfected with Cas9 mRNA premixed with gRNA and equal molar concentrations of NPTII mRNA. A mixture of 5×10⁴ protoplasts re-suspended in 200 μl MMG solution was gently mixed with RNA mixture and 50 μl of freshly prepared PEG solution (40% [w/v] PEG 4000; Fluka, #81240, 0.4 M mannitol (0.2 M in case of mannitol) and 0.1 M CaCl₂), and then incubated at 25° C. (in ice in case of tomato) for 10 min (15 min in case of Arabidopsis). After incubation, 1 ml W5 solution (154 mM NaCl, 125 mM CaCl₂, 5 mM KCL, 5 mM glucose; pH 5.8) was added slowly. The resulting solution was mixed well by inverting the tube. Protoplasts were pelleted by centrifugation at 70 g for 3 min and re-suspended gently in 1 ml (100 μl in case of GFP observation) W5 solution. For observation, the protoplasts were transferred into standard microscope slide (sealed between slide and coverslip with vaseline in case of GFP observation) and cultured under dark conditions at 25° C. for 48 h.

Protoplast Selection and Regeneration.

RNA-transfected protoplasts were re-suspended in 25 μl K3 liquid culture medium, embedded with 225 μl 1:1 mix of K3:H solid medium containing 0.6% Sea Plaque™ agarose (melted in a microwave oven and kept in a water bath at 40-45° C.), and then placed in darkness at 25° C. After 0-12 h, the protoplasts embedded in agarose were overlaid with 15 ml 1:1 mix of K3:H liquid medium (add 50 mg/L kanamycin in case of), and cultured at 25° C. in darkness followed by 6 days in continuous dim light (5 μmol m⁻² s⁻¹), where first and multiple cell divisions occur. Cut the agarose containing the dividing protoplasts into quadrants and place these in 15 ml medium A in a 10-cm plastic culture vessel. The cultures were incubated at 24° C. in continuous dim light on a rotary shaker at 80 rpm.

Rooting, Transfer to Soil and Hardening of Tobacco.

To regenerate whole plants, protoplast-derived resistant colonies (2-3 mm in diameter) were transferred onto MS medium containing 0.1 mg/L NAA and 1 mg/L 6-BA solidified with 0.6% (w/v) agarose (supplemental data) in 9 cm culture vessels and kept for 2-4 weeks at 25° C. under 16 h/day light. When shoots reach a size of 3-4 cm, they were be cut off and transferred to tubes containing 0.75% (w/v) agar-solidified MS medium. Roots will form in 1-3 weeks after transfer. Plantlets developed from adventitious shoots were subjected to acclimation, transplanted to potting soil, and maintained in a growth chamber at 25° C. under natural light.

Besides the use of protoplasts, a PDS-1000/He biolistic device (Bio-Rad #165-2257) was also used to deliver RNA-coated gold particles to target cells. The following bombardment conditions were used: a) RNA was precipitated onto 1-μm gold microcarriers; b) 1100 psi rupture disk was used, the gap between rupture disk retaining cap and macrocarrier was 0.9 cm; c) target cells were 5 cm below the microcarrier assembly shelf; and d) macrocarrier travel distance was 11 mm.

T7E1 Assay.

Genomic DNA was isolated from protoplasts or calli using CTAB (cetyl trimethylammonium bromide) method. The target DNA region was PCR amplified and used for the T7E1 assay. Briefly, PCR products were denatured at 95° C. for 10 minutes and then cooled down slowly to 25° C. using a thermal cycler. The annealed PCR products were incubated with 5 units of T7 endonuclease I (NEB) for 20 mins at 37° C. followed by visualization in gels through electrophoresis.

RGEN-RFLP.

The RNA-guided endonucleases-restriction fragment length polymorphism (RGEN-RFLP) assay was used with slight modification. Before each use, sgRNAs were incubated for 3 minutes in 95° C. in a PCR tube followed by immediately water/ice bath for 2 minutes to obtain pure monomers. Approximately 300-400 ng PCR products were incubated in 1×NEB buffer 3 for 60 min at 37° C. together with 1 μg Cas9 protein and 750 ng sgRNA in a 10 μl reaction. RNase A (4 μg) was then added to the reaction mixture and incubated at 37° C. for 30 mins to destroy RNA followed by addition of 6× stop solution (30% glycerol, 1.2% SDS, 100 mM EDTA) to stop the reaction. DNA products were visualized in 2.5% agarose gels.

Targeted genomic sequencing. The genomic region flanking the on-target and potential off-target sites were PCR amplified from genomic DNA. Then the PCR products were cloned into pGEM®-T vector (promega), followed by sequencing. For each target site, 15-30 independent colonies were chosen for sequencing. DNA sequence alignment was performed to compare targeted locus with wild-type sequence.

### Example 1

Cas9 mRNA (SEQ ID NO: 90) and guide RNA (gRNA) were obtained by in vitro transcription. To determine the optimal ratio of Cas9 mRNA and gRNA in transfecting protoplasts, the modified single strand assay (SSA) method was used. SSA is commonly used to test gRNA activity using green fluorescent protein (GFP) reporter. By keeping the SSA reporter vector at a constant level, the amount of Cas9 mRNA and gRNA were varied. It was found that a 1:20 molar ratio of Cas9 mRNA and gRNA provided the best results.

In stable transformation, a selectable marker gene provides a significant advantage for transformed cells to grow in the presence of selection reagent (e.g. kanamycin). Selectable marker genes are often built into engineered DNA plasmids used for genetic transformation, it is a major factor for successful genetic manipulation. To avoid stable integration of the marker gene into plant genome, a short-term selection system was developed. NPTII mRNA was obtained from in vitro transcription and introduced into tobacco protoplasts. Two control conditions (without adding NPTII mRNA) were tested; numerous colonies were formed without kanamycin (FIGS. 1A-1E, Control+K0), and no colony was observed when 50 mg/L kanamycin was added to the culture media (FIGS. 1A-1E, Control+K50). After NPTII mRNA was added, colonies were formed in the presence of 50 mg/L kanamycin (FIGS. 1A-1E, NPTII transcripts+K50). The results showed that the short-term selection system is effective. It should be noted that for such a short-term selection system, too short a selection time (e.g. 2 days) is not enough to inhibit cell growth, while extended selection inhibits all the cells. In this example, 6 day selection was used. The short-term selection offers an important advance over stable selection, since the marker gene is not integrated in the host genome and thus obviates the need to remove the selection cassette in the progeny.

### Example 2

To examine the effectiveness of combining the selection system with Cas9 mRNA and gRNA, two genes with obvious phenotypes were tested. One target is the phytoene desaturase (PDS) gene (SEQ ID NO: 92 in tobacco, CDS in SEQ ID NO: 93; PDS-like gene in SEQ ID NO: 94, CDS of SEQ ID NO: 94 in SEQ ID NO: 95). Disruption of the PDS gene results in albino plants due to the impairment of chlorophyll and carotenoid biosynthesis. The other target is the LAM1 gene (SEQ ID NO: 96 in tobacco, CDS in SEQ ID NO: 97), mutation of LAM1 blocks the formation of lamina and the lam1 mutant in Nicotiana sylvestris exhibits a bladeless phenotype.

To target the PDS gene, Cas9 mRNA (SEQ ID NO: 90), gRNA to target the PDS gene and NPTII mRNA (SEQ ID NO: 1) were introduced into tobacco protoplasts by PEG treatment. The results showed that many albino colonies were obtained after a short-term kanamycin selection (FIGS. 2A-2B). Albino plantlets were regenerated from these colonies (FIGS. 2C-2E). DNA was isolated from albino plantlets, sequencing results demonstrated that nucleotide additions and deletions indeed occurred in the target region of the PDS gene (FIGS. 3A-3C). The frequency of targeted homozygous mutagenesis reached 88% (Table 1), indicating this DNA free system is very effective in generating mutations. With selection, the frequency was more than 8 fold higher than that without selection (Table 1). This method proved the feasibility of mRNA-mediated transformation in plant cells and allows the production of non-transgenic plants. There is no need to segregate out the Cas9 elements and the marker genes.

TABLE 1
Frequency of mutagenesis in the PDS gene after protoplast transfection
and kanamycin selection. K50: 50 mg/l kanamycin selection for 6 days.
Data are mean ± standard deviation.

| | | Percentage (%) of albino calli/ |
|---|---|---|
| mRNA | K50 | total calli |
| − | − | 0.0 ± 0.0 |
| − | + | 0.0 ± 0.0 |
| Cas9, NPTII and PDS gRNA | − | 10.5 ± 7.2 |
| Cas9, NPTII and PDS gRNA | + | 88.6 ± 0.9 |

### Example 3

Similarly, to target the LAM1 gene, Cas9 mRNA (SEQ ID NO: 90), gRNA to target the LAM1 gene (one gRNA for Site 1 in FIG. 5A and one gRNA for Site 2 in FIG. 5A) and NPTII mRNA (SEQ ID NO: 1) were introduced into tobacco protoplasts by PEG treatment. Compared to wild-type control, the mutants showed bladeless phenotype (FIGS. 4A-4F). Sequencing results confirmed that nucleotide additions and deletions occurred in the target region of the LAM1 gene (FIGS. 5A-5B). The frequency of targeted homozygous mutagenesis reached 86% (Table 2). With selection, the frequency was more than 10 fold higher than that without selection (Table 2).

TABLE 2
Frequency of mutagenesis in the LAM1 gene after protoplast transfection
and kanamycin selection. K50: 50 mg/l kanamycin selection for 6 days.
Data are mean ± standard deviation.

| | | Percentage (%) of bladeless |
|---|---|---|
| mRNA | K50 | shoots/total shoots |
| − | − | 0.0 ± 0.0 |
| − | + | 0.0 ± 0.0 |
| Cas9, NPTII and LAM1 gRNA | − | 7.7 ± 2.0 |
| Cas9, NPTII and LAM1 gRNA | + | 86.5 ± 5.6 |

### Example 4

To target the PDS gene and the LAM1 gene simultaneously, Cas9 mRNA (SEQ ID NO: 90), PDS gRNA, LAM1 gRNA for Site 2 and NPTII mRNA (SEQ ID NO: 1) were introduced into tobacco protoplasts by PEG treatment. Double pds/lam1 mutants were readily obtained (FIGS. 6A-6E). The frequency of double mutants reached 74%.

In addition to tobacco, similar systems can be developed in other species, such as Arabidopsis, tomato, Medicago and rice. Preliminary results show that introduction of Cas9 mRNA and gRNA into cells of these species is effective in creating mutations.

The generation of specific, desired mutant plants without transgenesis will avoid most of the regulatory hurdles that inhibit the use of modern molecular techniques in producing and releasing improved cultivars.

The foregoing description of specific embodiments of the present disclosure has been presented for purpose of illustration and description. The exemplary embodiments were chosen and described in order to best explain the principles of the disclosure and its practical application, to thereby enable others skilled in the art to best utilize the subject matter and various embodiments with various modifications are suited to the particular use contemplated.

What is claimed is:

1. A method for selecting cells carrying a transfected nucleic acid comprising the steps of:
exposing a plurality of cells to a first RNA molecule under conditions sufficient to promote transfection of the first RNA molecule into one or more of the plurality of cells, wherein the first RNA molecule or a peptide or protein encoded by the first RNA molecule permits selection of the one or more of the plurality of cells transfected with the first RNA molecule based on a first selectable property; and
selecting the one or more cells of the plurality of cells transfected with the first RNA molecule based on the first selectable property.

2. The method of claim 1 further comprising the step of exposing the plurality of cells to a selection agent, the selection agent being sufficient to kill or reduce proliferation of the plurality of cells that are not transfected with the first RNA molecule before selecting the one or more cells of the plurality of cells transfected with the first RNA molecule, wherein the peptide or protein encoded by the first RNA molecule confers resistance to the selection agent as the first selectable property.

3. The method of claim 1 wherein the first selectable property is a visually detectable difference between the one or more of the plurality of cells transfected with the first RNA molecule and the plurality of cells that are not transfected with the first RNA molecule.

4. The method of claim 1 further comprising the step of exposing the plurality of cells to a starvation condition before selecting one or more cells of the plurality of cells based on the first selectable property, wherein the peptide or protein encoded by the first RNA molecule allows the one or more of the plurality of cells transfected with the first RNA molecule to survive under the starvation condition as the first selectable property.

5. The method of claim 1 wherein the peptide or protein encoded by the first RNA molecule impairs cellular growth due to intracellular production of a toxic agent as the first selectable property.

6. The method of claim 1 further comprising the step of exposing the plurality of cells to a non-toxic agent before selecting one or more cells of the plurality of cells based on the first selectable property, wherein the peptide or protein encoded by the first RNA molecule converts the non-toxic agent to a toxic agent.

7. The method of claim 1 further comprising the step of exposing the plurality of cells to a selection agent, the selection agent being sufficient to kill or reduce proliferation of the plurality of cells that are not transfected with the first RNA molecule before selecting the one or more cells of the plurality of cells transfected with the first RNA molecule, wherein the first RNA molecule is a silencing RNA (siRNA) that inhibits or reduces the expression of a gene in the plurality of cells that confers susceptibility to a selection agent.

8. The method of claim 1 wherein the plurality of cells are plant cells.

9. The method of claim 8 wherein the plant cells are derived from Arabidopsis thaliana, tobacco, hybrid aspen, muskmelon, potato, tomato, rice, maize, finger millet, soybean, alfalfa, rye, oat, wheat, triticale, hairy vetch, common vetch, white clover, red clover, radish, cotton, sugarcane, sugarbeet, tall fescue, ryegrass, switchgrass, sorghum, peanut, sunflower, canola, flax and barley.

10. The method of claim 8 wherein the plant cells are protoplasts or calluses.

11. The method of claim 2 wherein the selection agent is an antibiotic or herbicide.

12. The method of claim 11 wherein the selection agent is selected from the group consisting of kanamycin, geneticin, paromomycin, hygromycin, phosphinothricin, glyphosate, peptide deformylase, actinonin, tunicamycin, sulfonyl urea or imidazolinone herbicides, cyanamide, dalapon, organophosphorous pesticides, 2-deoxyglucose, fluridone, norflurazon, flurtamone, butafenacil, dinitroaniline or phosphoroamidate herbicides, and L-glutamate-1-semialdehyde.

13. The method of claim 2 wherein the peptide or protein encoded by the first RNA molecule is selected from the group consisting of NPTII, hph, bar, EPSPS, At-WBC19, DEF2, GPT, gox, gat4061, gat4621, pat, galT, mutant acetolactate/acetohydroxyacid synthase, cah, GSA, dehl, oph, DOG^{R}1, human P450, mutated pds, ppo, and Tub1.

14. The method of claim 2 wherein the step of exposing the plurality of cells to the selection agent is for a duration of from 1 day to 45 days.

15. The method of claim 1 wherein the conditions sufficient to promote transfection comprise exposing the cells to polyethylene glycol, electroporation or particle bombardment.

16. The method of claim 1 further comprising exposing the plurality of cells to a second RNA molecule and a third RNA molecule in addition to the first RNA molecule under conditions sufficient to promote transfection of the second RNA molecule and third RNA molecule, wherein the second RNA molecule encodes Cas9, wherein the third RNA molecule is guide RNA (gRNA), wherein the gRNA comprises a scaffold sequence for Cas9 binding and a target sequence, wherein the target sequence defines the genomic region of the plurality of cells to be edited, wherein the second RNA molecule and third RNA molecule are sufficient to edit a genomic region of one or more of the plurality of cells, and wherein the plurality of cells are plant cells.

17. A genetically-modified plant produced using the method of claim 16.

18. A seed produced by the genetically-modified plant of claim 17.

19. A kit comprising:
a first RNA molecule, wherein the first RNA molecule or a peptide or protein encoded by the first RNA molecule is sufficient to confer resistance to a selection agent;
the selection agent, wherein the selection agent is sufficient to inhibit or reduce proliferation of a plurality of cells in the absence of the first RNA molecule; and
a transfection reagent.

20. The kit of claim 20 further comprising:
a second RNA molecule; and
a third RNA molecule,
wherein the second RNA molecule encodes Cas9, wherein the third RNA molecule is guide RNA (gRNA), and wherein the gRNA comprises a scaffold sequence for Cas9 binding and a target sequence.
